(19) United States
(12) Patent Application Publication
(10) Pub. No.: US 2025/0011287 A1
(43) Pub. Date: Jan. 9, 2025

(54) FLUORINE-CONTAINING COMPOUND AND CONTRAST AGENT

(71) Applicant: TDK CORPORATION, Tokyo (JP)

(72) Inventor: Naoko YANAI, Tokyo (JP)

(73) Assignee: TDK CORPORATION, Tokyo (JP)

(21) Appl. No.: 18/700,598

(22) PCT Filed: Aug. 12, 2022
(86) PCT No.: PCT/JP2022/030718

§ 371 Date: Apr. 11, 2024

(30) Foreign Application Priority Data
Nov. 11, 2021 (JP) 2021-183815

(51) Int. Cl. C07D 211/94; A61K 49/06

(52) U.S. Cl. CPC C07D 211/94; A61K 49/06

(57) ABSTRACT

This fluorine-containing compound is represented by Formula (1). R¹, R², R³, and R⁴ are an alkyl group which has 1 to 10 carbon atoms and which is substituted with a substituent not containing a fluorine atom or is unsubstituted. X is —(CH₂)— or —(CH₂)ₘ—O—(CH₂)—; Y is —CZ₃ or —C(CH₃)Z₂; and Z is —CH₂—O—C(CF₃)₃.

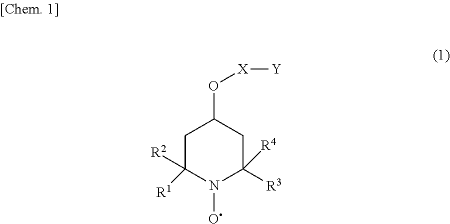

### TECHNICAL FIELD

The present invention relates to a fluorine-containing compound and a contrast agent.

The present application claims priority on Japanese Patent Application No. 2021-183815 filed on Nov. 11, 2021, the content of which is incorporated herein by reference.

### BACKGROUND ART

Magnetic resonance imaging (hereinafter sometimes referred to as “MRI”) diagnosis is widely used in the medical field for both basic research and clinical applications as one of imaging diagnostic methods along with X-ray diagnosis and ultrasonic (US) diagnosis.

Currently, ¹H-MRI, which uses protons (¹H) as a detection nucleus, is used in medical MRI. In ¹H-MRI, the magnetic environment of water molecules present in a living body is captured to be formed into an image. There is a difference in the magnetic environment of protons between diseased tissue and normal tissue in a living body. This appears as a difference in ¹H-MRI and is taken as diagnostic information. Furthermore, water molecules are present in almost all regions in a living body. Therefore, ¹H-MRI can be used for whole-body imaging.

In addition to ¹H, examples of nuclides that can be detected by MRI include ¹⁹F, ²³Na, ³¹P, ⁵N, and ¹³C. In MRI using these elements as a detection nucleus, information which is different from that of ¹H-MRI is obtained for each element.

Among them, MRI using ¹⁹F as a detection nucleus is expected to be used as a next-generation diagnostic method following ¹H-MRI diagnosis. This is because fluorine is an inexpensive element with a 100% natural abundance, the detection sensitivity of ¹⁹F is as high as 83% of ¹H, and the gyromagnetic ratio of ¹⁹F is close to that of protons; and thereby, it is possible to obtain images by a conventional ¹H-MRI device.

Furthermore, ¹⁹F, which can be detected by MRI, is barely present in living bodies. Therefore, by using a compound containing a fluorine atom as a contrast agent, ¹⁹F-MRI diagnosis using ¹⁹F as a tracer is possible. For example, positional information on lesion areas can be obtained from ¹⁹F-MRI by using, as a contrast agent, a fluorine compound that accumulates by recognizing endogenous changes caused by a disease. This method is useful for diagnosing lesion areas that do not cause morphological changes, which could not be detected by conventional imaging diagnostic methods.

Currently, nuclear medicine techniques are available as a method for obtaining image information specific to lesion areas. Nuclear medicine techniques use radiopharmaceuticals that utilize radioactive isotopic elements. Specifically, as nuclear medicine techniques, there are a positron emission tomography (PET) test, and a single photon emission computed tomography (SPECT) test. However, nuclear medicine techniques have problems such as the requirement of large-scale equipment for synthesizing radioactive isotopes and the risk of exposure to radiation.

¹⁹F-MRI diagnosis does not cause the above-mentioned problems in nuclear medicine techniques. In addition, in ¹⁹F-MRI diagnosis, by extracting information such as a chemical shift, diffusion, and relaxation time, not only the positional information on lesion areas but also more items of diagnostic information can be obtained. Furthermore, by simultaneously imaging using ¹⁹F-MRI and ¹H-MRI in a single diagnosis and overlapping each image, it is possible to obtain useful diagnostic information that includes both anatomical information and functional information.

Examples of contrast agents for MRI diagnosis that use fluorine as a detection nucleus include those disclosed in Patent Document 1 and Patent Document 2.

Patent Document 1 discloses particles of a lactic acid-glycolic acid copolymer (PLGA) containing a perfluoro crown ether and a gadolinium complex. Furthermore, Patent Document 2 discloses a fluorine-containing porphyrin complex and a contrast agent compound which can be used in MRI using fluorine as a detection nucleus.

However, since the contrast agents disclosed in Patent Document 1 and Patent Document 2 contain metal ions, there are concerns about safety in a living body.

Furthermore, Patent Document 3 discloses a compound having a nitroxide covalently bonded to a fluorine-containing compound. However, because the fluorine-containing compound disclosed in Patent Document 3 is easily reduced by a reducing agent such as ascorbic acid (refer to Non-Patent Document 1, for example), there is a problem of stability in a living body.

Conventional contrast agents for MRI diagnosis that use fluorine as a detection nucleus are not agents that can provide highly sensitive MRI and that have high stability in a living body.

### CITATION LIST

### Patent Document

[Patent Document 1]
Published Japanese Translation No. 2015-534549 of the PCT International Publication
[Patent Document 2]
Japanese Unexamined Patent Application, First Publication No. H11-217385
[Patent Document 3]
U.S. Pat. No. 5,362,477

### Non-Patent Document

[Non-Patent Document 1]
ACS Central Science, 2017, 3, Pages 800 to 811

### SUMMARY OF INVENTION

### Technical Problem

The present invention has been made in view of the above-mentioned circumstances, and an object of the present invention is to provide a fluorine-containing compound which is highly stable in a living body and which makes it possible to obtain highly sensitive magnetic resonance images by being used as a material for a contrast agent for magnetic resonance imaging diagnosis that uses fluorine as a detection nucleus.

Another object of the present invention is to provide a contrast agent for magnetic resonance imaging diagnosis that uses fluorine as a detection nucleus, and the contrast agent for magnetic resonance imaging diagnosis contains the fluorine-containing compound of the present invention, is highly stable in a living body, and makes it possible to obtain highly sensitive images.

### Solution to Problem

[1] A fluorine-containing compound represented by General Formula (1) below.

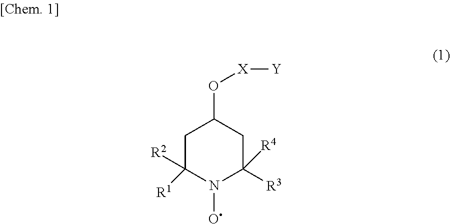

(In General Formula (1), R¹, R², R³, and R⁴ are each independently an alkyl group which has 1 to 10 carbon atoms and which is substituted with a substituent not containing a fluorine atom or is unsubstituted, X is represented by General Formula (2-1) or (2-2) below, and Y is represented by General Formula (3-1) or (3-2) below.)

—(CH₂)— (2-1)

—(CH₂)ₘ—O—(CH₂)— (2-2)
(In Formula (2-2), m is an integer of 1 to 12, and —(CH₂)ₘ— in Formula (2-2) is bonded to an oxygen atom in Formula (1).)

—CZ₃ (3-1)
(In Formula (3-1), Z is —CH₂—O—C(CF₃)₃.)

—C(CH₃)Z₂ (3-2)
(In Formula (3-2), Z is —CH₂—O—C(CF₃)₃.)

[2] The fluorine-containing compound according to [1], in which R¹, R², R³, and R⁴ in General Formula (1) above are each independently an alkyl group which has 1 to 5 carbon atoms and which is substituted with a substituent not containing a fluorine atom or is unsubstituted.

[3] The fluorine-containing compound according to [1] or [2], in which Y is represented by General Formula (3-1) above.

[4] The fluorine-containing compound according to any one of [1] to [3], in which X is represented by General Formula (2-2) above.

[5] The fluorine-containing compound according to any one of [1] to [4], in which R¹, R², R³, and R⁴ in General Formula (1) above are each independently a methyl group or an ethyl group.

[6] The fluorine-containing compound according to any one of [1] to [5], in which the fluorine-containing compound is used as a contrast agent for magnetic resonance imaging diagnosis using fluorine as a detection nucleus.

[7] A contrast agent for magnetic resonance imaging diagnosis which uses fluorine as a detection nucleus, the contrast agent for magnetic resonance imaging diagnosis containing: the fluorine-containing compound according to any one of [1] to [6].

### Advantageous Effects of Invention

A fluorine-containing compound according to one aspect of the present invention is a compound represented by General Formula (1) above. Therefore, stability in a living body is high. Furthermore, when the fluorine-containing compound according to one aspect of the present invention is used as a material for a contrast agent for magnetic resonance imaging diagnosis using fluorine as a detection nucleus, highly sensitive magnetic resonance images can be obtained.

The contrast agent according to one aspect of the present invention contains the fluorine-containing compound according to one aspect of the present invention. Therefore, the contrast agent according to one aspect of the present invention has high stability in a living body. Furthermore, when the contrast agent according to one aspect of the present invention is used as a contrast agent for magnetic resonance imaging diagnosis using fluorine as a detection nucleus, highly sensitive magnetic resonance images can be obtained.

### BRIEF DESCRIPTION OF DRAWINGS

FIG. 1 is a ¹⁹F spin-lattice relaxation time (T1) weighted image of ¹⁹F-MRI in Example 1 (compound 11), Example 3 (compound 13), and Comparative Example 1 (compound A1).

FIG. 2 is a ¹⁹F spin-lattice relaxation time (T1) weighted image of ¹⁹F-MRI in Example 1 (compound 11), Example 3 (compound 13), and Comparative Example 1 (compound A1), and is a photograph showing the positions of Examples 1 and 3 and Comparative Example 1 on the image shown in FIG. 1.

### DESCRIPTION OF EMBODIMENTS

Hereinbelow, a fluorine-containing compound and a contrast agent of the present embodiment will be described in detail.

### [Fluorine-Containing Compound]

The fluorine-containing compound of the present embodiment is represented by General Formula (1) below.

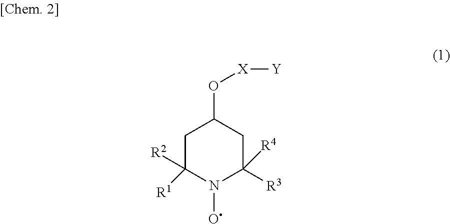

(In General Formula (1), R¹, R², R³, and R⁴ are each independently an alkyl group which has 1 to 10 carbon atoms and which is substituted with a substituent not containing a fluorine atom or is unsubstituted, X is represented by General Formula (2-1) or (2-2) below, and Y is represented by General Formula (3-1) or (3-2) below.)

—(CH₂)— (2-1)

—(CH₂)ₘ—O—(CH₂)— (2-2)
(In Formula (2-2), m is an integer of 1 to 12, and —(CH₂)ₘ— in Formula (2-2) is bonded to an oxygen atom in Formula (1).)

—CZ₃ (3-1)
(In Formula (3-1), Z is —CH₂—O—C(CF₃)₃.)

—C(CH₃)Z₂ (3-2)
(In Formula (3-2), Z is —CH₂—O—C(CF₃)₃.)

When the contrast agent containing the fluorine-containing compound of the present embodiment is used as a contrast agent for MRI diagnosis using fluorine as a detection nucleus, the reason why stability in a living body is high and highly sensitive magnetic resonance images (MRIs) can be obtained will be described.

In order to obtain ¹⁹F-MRI with high sensitivity, it is preferable to use a fluorine-containing compound that has a short ¹⁹F spin-lattice relaxation time (T1) as a fluorine-containing compound contained in the contrast agent. The shorter T1 of the fluorine-containing compound, the shorter the repetition time can be set. Therefore, the amount of signals obtained per unit time increases, and a highly sensitive image can be obtained. On the other hand, when a ¹⁹F spin-spin relaxation time (T2) of the fluorine-containing compound is too short, a signal intensity decreases.

The ¹⁹F spin-lattice relaxation time (T1) and the ¹⁹F spin-spin relaxation time (T2) of the fluorine-containing compound are affected by a paramagnetic relaxation enhancement (PRE) effect. The PRE effect is a phenomenon in which T1 and T2 of an MRI observation nucleus near unpaired electron spins are shortened due to the unpaired electron spins of a paramagnetic material.

The PRE effect is inversely proportional to the sixth power of the distance between a paramagnetic material and an MRI observation nucleus (fluorine atom in the present embodiment) relaxed by the paramagnetic material. Therefore, in the fluorine-containing compound represented by Formula (1) of the present embodiment, T1 and T2 become shorter as the distance between a nitroxide radical, which is a paramagnetic material, and a fluorine atom, becomes shorter. In the fluorine-containing compound represented by Formula (1), a substituent (X-Y in Formula (1)) in which a fluorine atom is bonded to the terminal is bonded to the 4th-position carbon of a piperidine ring via an oxygen atom. Therefore, the distance between the nitroxide radical and the fluorine atom is appropriate, which makes T1 sufficiently short and makes it possible to secure a sufficient T2. Accordingly, by using the fluorine-containing compound represented by Formula (1) as a contrast agent for MRI diagnosis using fluorine as a detection nucleus, highly sensitive magnetic resonance images can be obtained.

Furthermore, unlike closed-shell species, organic radicals have a singly occupied molecular orbital (SOMO), which contains an unpaired electron, between the highest occupied molecular orbital (HOMO) and the lowest unoccupied molecular orbital (LUMO). The redox process of organic radicals corresponds to the process of electron transfer in SOMO. The reduction reaction of organic radicals by a reducing agent such as ascorbic acid is more likely to occur as an energy difference between the HOMO of the reducing agent and the SOMO of organic radicals becomes smaller. Therefore, the lower the SOMO energy level of organic radicals, the easier it is for the reduction to occur.

In the fluorine-containing compound represented by Formula (1) of the present embodiment, three carbon atoms are disposed between a nitrogen atom of the piperidine ring and the fluorine-atom-containing substituent represented by X-Y in Formula (1). Furthermore, the oxygen atom is bonded to the substituent represented by X-Y, and five or more carbon atoms are disposed between the oxygen atom and the fluorine atom. As a result, in the fluorine-containing compound represented by Formula (1), the nitroxide radical and the fluorine atom are disposed at sufficiently distant positions, and the nitroxide radical is less susceptible to electronic influences from the fluorine atom. Therefore, in the fluorine-containing compound represented by Formula (1), a decrease in the SOMO energy level of the nitroxide radical due to the fluorine atom, which is an electron-withdrawing group, does not occur. Accordingly, the energy difference between the SOMO of the nitroxide radical in the fluorine-containing compound of the present embodiment and the HOMO of a reducing agent such as ascorbic acid is sufficiently large. Therefore, the fluorine-containing compound represented by Formula (1) is less likely to be reduced in a living body; and thereby, stability in a living body becomes high.

Furthermore, because the fluorine-containing compound represented by Formula (1) of the present embodiment is a non-metallic compound that does not contain metals, safety in a living body is higher than a contrast agent containing metal ions. Therefore, the fluorine-containing compound of the present embodiment is suitable as a material for a contrast agent for magnetic resonance imaging diagnosis using fluorine as a detection nucleus.

Furthermore, the fluorine-containing compound represented by Formula (1) of the present embodiment has a structure similar to 4-hydroxy-2,2,6,6-tetramethylpiperidine-1-oxyl free radical (TEMPOL), which is a compound having a piperidine ring and is highly safe in a living body. Therefore, for example, the fluorine-containing compound represented by Formula (1) of the present embodiment is presumed to have higher stability in a living body as compared to a fluorine-containing compound having a pyrrolidine ring.

In the fluorine-containing compound represented by Formula (1) of the present embodiment, R¹, R², R³, and R⁴ are each independently an alkyl group which has 1 to 10 carbon atoms and which is substituted with a substituent not containing a fluorine atom or is unsubstituted, and are each independently preferably an alkyl group which has 1 to 5 carbon atoms and which is substituted with a substituent not containing a fluorine atom or is unsubstituted. Since R¹, R², R³, and R⁴ are substituted or unsubstituted alkyl groups having 1 to 10 carbon atoms, the fluorine-containing compound represented by Formula (1) can be easily synthesized. Furthermore, when R¹, R², R³, and R⁴ are substituted or unsubstituted alkyl groups having 2 to 10 carbon atoms, they become appropriately bulky; and thereby, it is possible to prevent a reducing agent from approaching the nitroxide radical. The number of carbon atoms in the above-mentioned alkyl group is preferably 5 or less because the synthesis of the fluorine-containing compound represented by Formula (1) becomes even easier.

When R¹, R², R³, and R⁴ contained in the fluorine-containing compound represented by Formula (1) have a substituent that does not contain a fluorine atom, for example, it is possible to use a methyl group or an ethyl group as the substituent.

Specifically, R¹, R², R³, and R⁴ in the fluorine-containing compound represented by Formula (1) of the present embodiment are preferably methyl groups or ethyl groups, and are more preferably ethyl groups because a SOMO energy level becomes high.

R¹, R², R³, and R⁴ contained in the fluorine-containing compound represented by Formula (1) may be different from each other, or some or all of them may be the same as each other. It is preferable that R¹, R², R³, and R⁴ be all the same because this facilitates the synthesis of the fluorine-containing compound represented by Formula (1).

In the fluorine-containing compound represented by Formula (1) of the present embodiment, X is represented by General Formula (2-1) or (2-2) above, and Y is represented by General Formula (3-1) or (3-2) above. Therefore, the fluorine-containing compound represented by Formula (1) has a sufficiently large number of fluorine atoms, and the distance between the nitroxide radical and the fluorine atom is appropriate; and thereby, T1 becomes sufficiently short and it is possible to secure a sufficient T2. Accordingly, by using the fluorine-containing compound represented by Formula (1) as a contrast agent for MRI diagnosis using fluorine as a detection nucleus, highly sensitive images can be obtained. Furthermore, since the distance between the nitroxide radical and the fluorine atom is appropriate, the nitroxide radical is less susceptible to electronic influences from the fluorine atom. Furthermore, since the substituent represented by X-Y is bulky, the approach of a reducing agent to the nitroxide radical is sterically blocked and prevented. Therefore, the fluorine-containing compound represented by Formula (1) is less likely to be reduced in a living body; and thereby, stability in a living body becomes high.

In the fluorine-containing compound represented by Formula (1), when X is represented by Formula (2-1), the distance between the nitroxide radical and the fluorine atom is appropriate. As a result, the nitroxide radical is less susceptible to electronic influences from the fluorine atoms; and thereby, a fluorine-containing compound has high stability in a living body. In addition, since the distance between the nitroxide radical and the fluorine atom does not become too long, which makes T1 sufficiently short, it is possible to obtain highly sensitive images by using the fluorine-containing compound as a contrast agent for MRI diagnosis using fluorine as a detection nucleus. Furthermore, the case where X is represented by Formula (2-1) is preferable because the synthesis is easier and productivity becomes excellent, as compared to the case where X is represented by Formula (2-2).

In the fluorine-containing compound represented by Formula (1), when X is represented by Formula (2-2), in in Formula (2-2) is an integer of 1 to 12, and thus the distance between the nitroxide radical and the fluorine atom is appropriate. As a result, the nitroxide radical is less susceptible to electronic influences from the fluorine atoms; and thereby, the fluorine-containing compound has higher stability in a living body. When m in Formula (2-2) is an integer of 12 or less, the distance between the nitroxide radical and the fluorine atom does not become too long; and thereby, T1 becomes sufficiently short, and thus it is possible to obtain highly sensitive images by using the fluorine-containing compound as a contrast agent for MRI diagnosis using fluorine as a detection nucleus. m in Formula (2-2) is more preferably 2 or 3 because the distance between the nitroxide radical and the fluorine atom does not become too long; and thereby, T1 becomes shorter.

In the fluorine-containing compound represented by Formula (1), Y is represented by Formula (3-1) or Formula (3-2). Therefore, Y contains two or three Z (—CH₂—O—C(CF₃)₃)'s.

Z has three trifluoromethyl groups (—CF₃) and thus contains nine fluorine atoms. Therefore, the fluorine-containing compound represented by Formula (1) has a sufficiently large number of fluorine atoms to exhibit a single ¹⁹F-MRI peak, and when the fluorine-containing compound is used as a contrast agent for MRI diagnosis using fluorine as a detection nucleus, a strong signal intensity is obtained; and thereby, it is possible to obtain highly sensitive images. The case where Y is Formula (3-1) is preferable because the number of fluorine atoms further increases; and thereby, it becomes easier to obtain highly sensitive images. Furthermore, the case where Y is Formula (3-1) is preferable because the fluorine-containing compound can be produced in fewer production steps as compared to the case where Y is Formula (3-2).

The combination of X and Y in the fluorine-containing compound represented by Formula (1) of the present embodiment is not particularly limited, but a combination in which X is Formula (2-1) and Y is Formula (3-1), a combination in which X is Formula (2-1) and Y is Formula (3-2), and a combination in which X is Formula (2-2) and Y is Formula (3-1) are preferable. Among the above-mentioned combinations, combinations in which Y is Formula (3-1) are particularly preferable. This is because since the fluorine-containing compound has a larger number of fluorine atoms, it becomes easier to obtain images having higher sensitivity when the fluorine-containing compound is used as a contrast agent for MRI diagnosis using fluorine as a detection nucleus.

Specifically, the fluorine-containing compound represented by Formula (1) is preferably any of fluorine-containing compounds represented by Formulas (11) to (20) below.

The compounds represented by Formulas (11) and (12) are fluorine-containing compounds in which X is Formula (2-1) and Y is Formula (3-1).

The compound represented by Formula (13) is a fluorine-containing compound in which X is Formula (2-1) and Y is Formula (3-2).

The compounds represented by Formulas (14) to (20) are fluorine-containing compounds in which X is Formula (2-2) and Y is Formula (3-1).

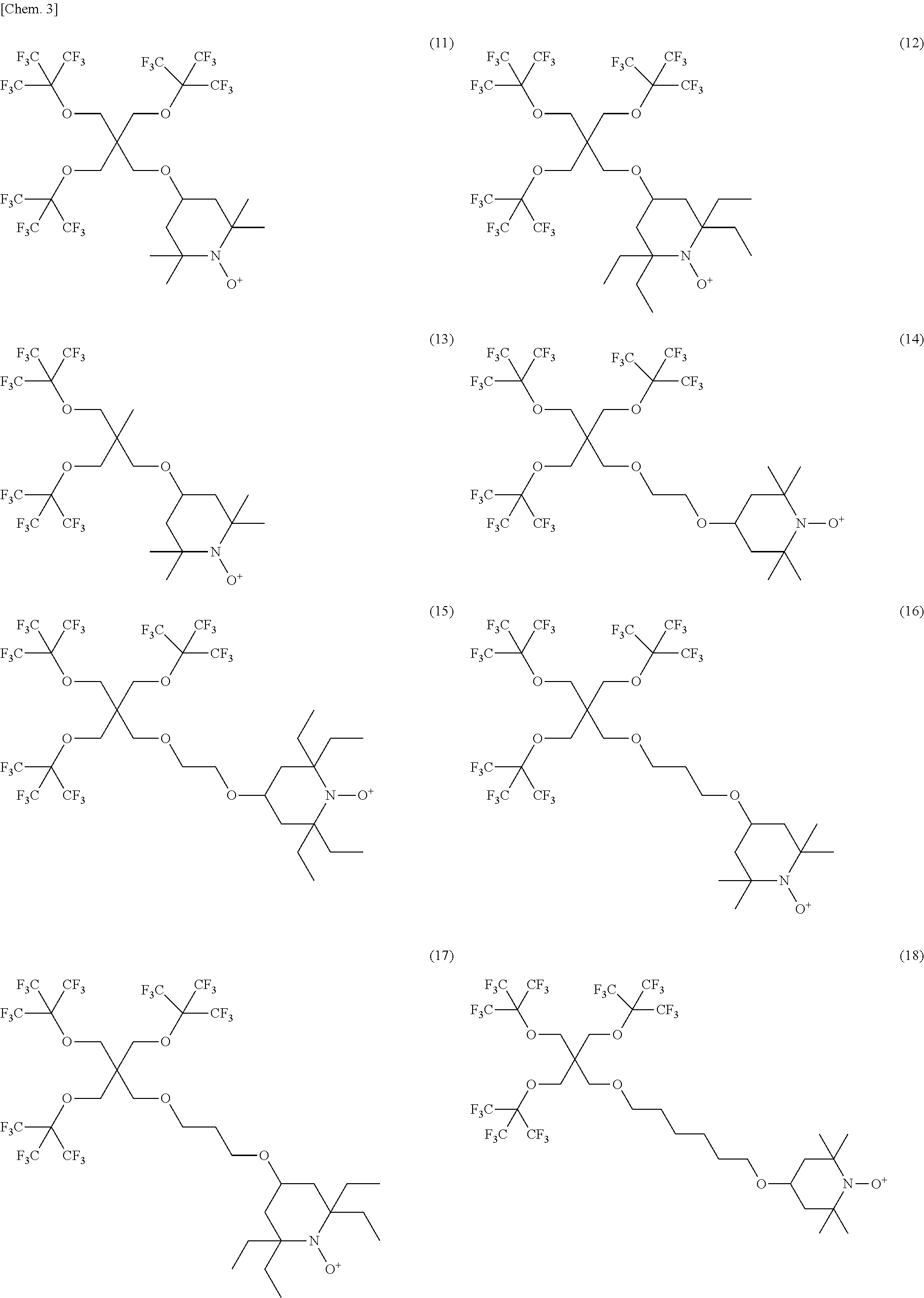

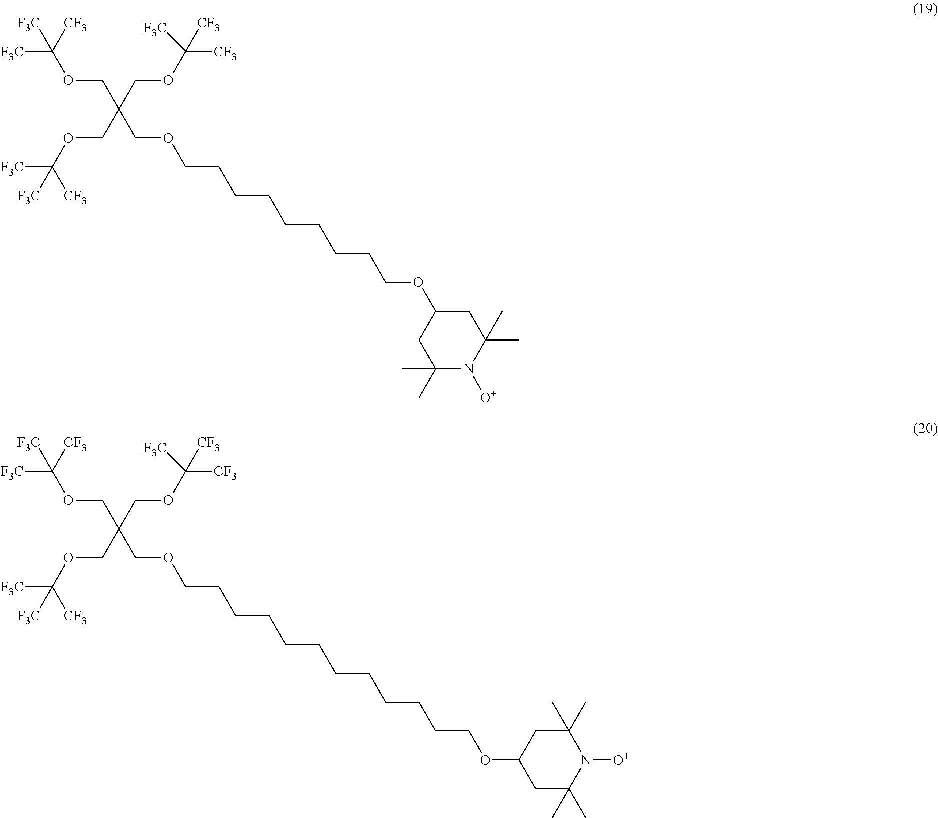 [SMILES: CC1(C)CC(OCCCCCCCCCOCC(COC(C(F)(F)F)(C(F)(F)F)C(F)(F)F)(COC(C(F)(F)F)(C(F)(F)F)C(F)(F)F)COC(C(F)(F)F)(C(F)(F)F)C(F)(F)F)CC(C)(C)N1[OH2+] ; CC1(C)CC(OCCCCCCCCCCCCOCC(COC(C(F)(F)F)(C(F)(F)F)C(F)(F)F)(COC(C(F)(F)F)(C(F)(F)F)C(F)(F)F)COC(C(F)(F)F)(C(F)(F)F)C(F)(F)F)CC(C)(C)N1[OH2+]]

### [Method for Producing Fluorine-Containing Compound]

Next, a method for producing the fluorine-containing compound of the present embodiment represented by Formula (1) will be described with reference to an example.

The method for producing the fluorine-containing compound of the present embodiment is not particularly limited, and production can be performed using a conventionally known production method.

For example, the fluorine-containing compound of the present embodiment represented by Formula (1) can be produced using the production method described below.

(Case where X is Formula (2-1))

First, a compound having a nitroxide radical is prepared, and in the nitroxide radical, R¹, R², R³, and R⁴ in the fluorine-containing compound represented by Formula (1) are bonded to the 2nd-position and the 6th-position of the piperidine ring, respectively, and a hydroxyl group is bonded to the 4th-position of the piperidine ring. This compound may be synthesized, or a commercially available one may be used. When synthesizing, for example, a known synthesis method in which 2,2,6,6-tetramethyl-4-piperidone is used as a raw material can be applied.

Furthermore, a halogen compound having a group corresponding to X-Y in the fluorine-containing compound represented by Formula (1) and having a halogeno group is prepared. Specifically, a halogen compound having either one of —CZ₃ (where Z in the formula is —CH₂—O—C(CF₃)₃) or —C(CH₃)Z₂ (where Z in the formula is —CH₂—O—C(CF₃)₃), and a group represented by r-(CH₂)— (where r in the formula is a halogeno group) is prepared. This halogen compound can be produced by a known method. For example, the halogen compound can be produced by a method in which a compound having two or three hydroxymethyl groups (—CH₂OH) and a halogenated alkyl group, and nonafluoro-tert-butanol are reacted with each other.

Thereafter, the halogeno group of the above-mentioned halogen compound is reacted with the hydroxyl group of the above-mentioned compound having the nitroxide radical. As a result, a group corresponding to X-Y is bonded to the oxygen atom bonded to the 4th-position of the piperidine ring.

By the above-described method, a fluorine-containing compound represented by Formula (1) in which X is Formula (2-1) can be obtained.

(Case where X is Formula (2-2))

Similarly to the case in which X is Formula (2-1), a compound having a nitroxide radical is prepared.

Next, a tetrahydropyranyl ether containing a group corresponding to —(CH₂)ₘ— in Formula (2-2) is prepared. Specifically, a compound having a group represented by r-(CH₂)ₘ—O— (in the formula, m is an integer of 1 to 12, and r is a halogeno group) at the 2nd-position of tetrahydropyran is prepared. This compound can be produced by a known method.

Next, the halogeno group of the above-mentioned tetrahydropyranyl ether is reacted with the hydroxyl group of the above-mentioned compound having a nitroxide radical to obtain a first intermediate compound. Thereafter, tetrahydropyran is removed from the first intermediate compound by a known method. This results in a second intermediate compound in which a group having a hydroxyl group at the terminal of a chain structure corresponding to —(CH₂)ₘ— is bonded to the oxygen atom bonded to the 4th-position of a piperidine ring.

Furthermore, a halogen compound having either one of —CZ₃ (where Z in the formula is —CH₂—O—C(CF₃)₃) or —C(CH₃)Z₂ (where Z in the formula is —CH₂—O—C(CF₃)₃), and a group represented by r-(CH₂)— (where r in the formula is a halogeno group) is prepared. This halogen compound can be produced by a known method. For example, the halogen compound can be produced by a method in which a compound having two or three hydroxymethyl groups (—CH₂OH) and a halogenated methyl group, and nonafluoro-tert-butanol are reacted with each other.

Thereafter, the halogeno group of the above-mentioned halogen compound is reacted with the hydroxyl group of the second intermediate compound. As a result, a group corresponding to X-Y is bonded to the oxygen atom bonded to the 4th-position of the piperidine ring.

By the above-described method, a fluorine-containing compound represented by Formula (1) in which X is Formula (2-2) can be obtained.

### “Contrast Agent”

A contrast agent of the present embodiment contains the fluorine-containing compound of the present embodiment. The contrast agent of the present embodiment is a contrast agent for magnetic resonance imaging diagnosis using fluorine as a detection nucleus.

The contrast agent of the present embodiment can be produced by a method of formulating the fluorine-containing compound of the present embodiment into a form such as a solid medication, a powder medication, and a liquid medication using a known formulation technique.

In addition to the fluorine-containing compound of the present embodiment, the contrast agent of the present embodiment may contain one or more additives used in known medications, such as excipients, stabilizers, surfactants, buffering agents, and electrolytes as necessary.

Since the contrast agent of the present embodiment contains the fluorine-containing compound of the present invention, stability in a living body is high. Furthermore, when the contrast agent of the present embodiment is used as a contrast agent for magnetic resonance imaging diagnosis using fluorine as a detection nucleus, highly sensitive magnetic resonance images can be obtained.

Although the embodiments of the present invention have been described in detail above, the configurations, the combinations thereof, and the like in each embodiment are merely examples, and additions, omissions, substitutions, and other alterations of the configuration may be made within a range not departing from the features the present invention.

### EXAMPLES

### Example 1

### (Synthesis of Compound 11)

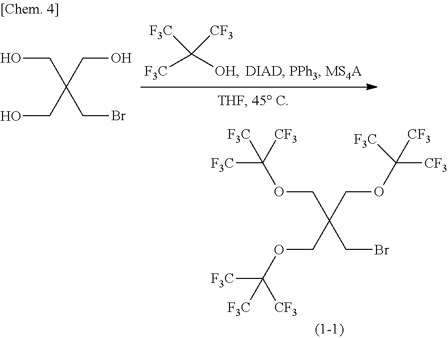

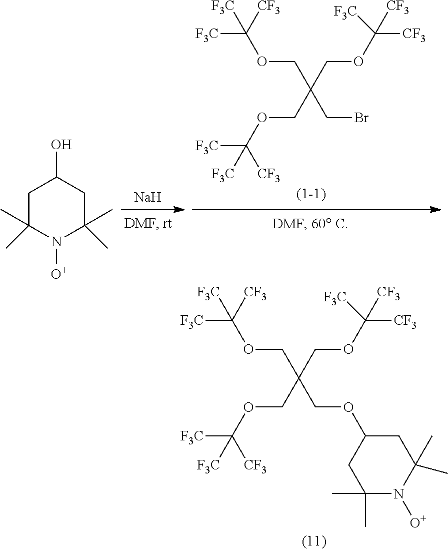

### Synthesis of 2-(3-bromo-2,2-bis(((1,1,1,3,3,3-hexafluoro-2-(trifluoromethyl)propan-2-yl)oxy)methyl)propoxy)-1,1,1,3,3,3-hexafluoro-2-(trifluoromethyl)propane (1-1)

Under an argon stream, 5.000 g (25.1 mmol) of 2-(bromomethyl)-2-(hydroxymethyl)-1,3-propanediol, 27.777 g (105.9 mmol) of triphenylphosphine (PPh₃), 10.000 g of Molecular Sieve (MS) 4A, and 130 ml of tetrahydrofuran (THF) were mixed and cooled in an ice bath. Thereto, 20.6 ml (105.9 mmol) of diisopropylazodicarboxylic acid ester (DIAD) was added dropwise over 10 minutes, and the mixture was stirred for 20 minutes. Furthermore, 25.000 g (105.9 mmol) of nonafluoro-tert-butanol was added at one time, and the mixture was stirred at 45° C. for 72 hours.

The reaction solution was filtered. Subsequently, the resultant was concentrated under reduced pressure, and purification was carried out by silica gel column chromatography (hexane:ethyl acetate=9:1) to obtain the target compound 2-(3-bromo-2,2-bis(((1,1,1,3,3,3-hexafluoro-2-(trifluoromethyl)propan-2-yl)oxy)methyl)propoxy)-1,1,1,3,3,3-hexafluoro-2-(trifluoromethyl)propane (1-1) (actual yield: 16.055 g, percent yield: 75%).

### Synthesis of 4-(3-((1,1,1,3,3,3-hexafluoro-2-(trifluoromethyl)propan-2-yl)oxy)-2,2-bis(((1,1,1,3,3,3-hexafluoro-2-(trifluoromethyl)propan-2-yl)oxy)methyl)propoxy)-2,2,6,6-tetramethylpiperidin-1-oxyl (11)

Under an argon stream, 10 ml of dimethylformamide (DMF) was added to 0.567 g (13.0 mmol) of 55% sodium hydride (NaH), and the mixture was stirred at room temperature for 10 minutes. Thereto, 20 ml of a dimethylformamide solution containing 2.239 g (13.0 mmol) of 4-hydroxy-2,2,6,6-tetramethylpiperidin-1-oxyl was added dropwise over 10 minutes, and the mixture was stirred at room temperature for 3 hours. Furthermore, 8.258 g (9.68 mmol) of 2-(3-bromo-2,2-bis(((1,1,1,3,3,3-hexafluoro-2-(trifluoromethyl)propan-2-yl)oxy)methyl)propoxy)-1,1,1,3,3,3-hexafluoro-2-(trifluoromethyl)propane (1-1) synthesized by the above-mentioned reaction and 60 ml of dimethylformamide was added in an ice bath, and the mixture was stirred at 60° C. for 16 hours.

Water was added to the reaction solution, and extraction was carried out with ethyl acetate. Thereafter, washing was carried out with a saturated sodium chloride aqueous solution, and the organic layer was dried with magnesium sulfate. The resultant was concentrated under reduced pressure. Subsequently, purification was carried out by silica gel column chromatography (hexane:ethyl acetate=9:1 to 4:1) to obtain the target 4-(3-((1,1,1,3,3,3-hexafluoro-2-(trifluoromethyl)propan-2-yl)oxy)-2,2-bis(((1,1,1,3,3,3-hexafluoro-2-(trifluoromethyl)propan-2-yl)oxy)methyl)propoxy)-2,2,6,6-tetramethylpiperidin-1-oxyl (11) (actual yield: 7.503 g, percent yield: 83%).

When the obtained compound was subjected to mass spectrometry, a peak was confirmed at m/z=944 (M⁺). From this result, it was confirmed that the synthesized compound was the compound represented by Formula (11). Furthermore, the degree of purity of the compound represented by Formula (11) confirmed by high-performance liquid chromatography (HPLC) was 98.1%.

### Example 2

### (Synthesis of Compound 12)

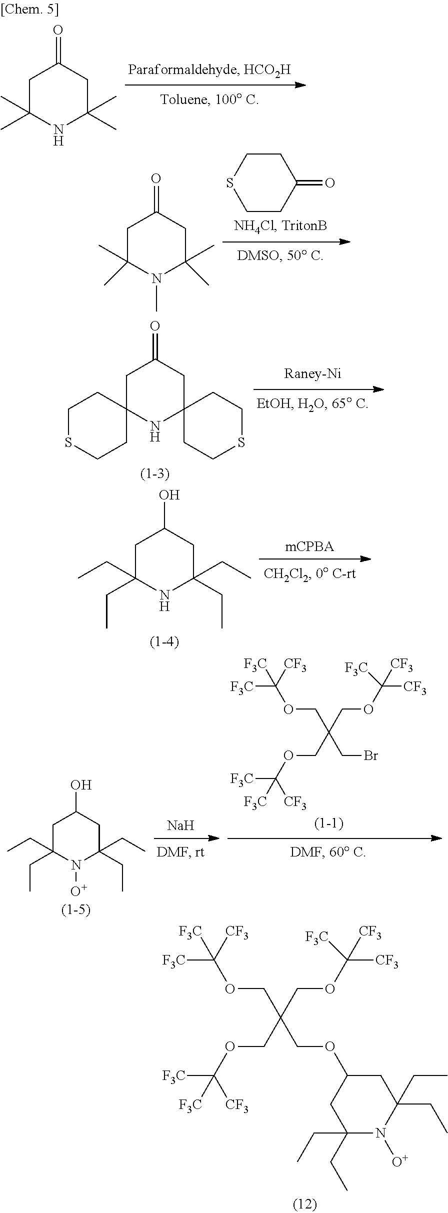

### Synthesis of 1,2,2,6,6-pentamethyl-4-piperidone (1-2)

Under an argon stream, 15.524 g (100 mmol) of 2,2,6,6-tetramethyl-4-piperidone, 23.288 g (150 mmol) of paraformaldehyde, and 100 ml of toluene were mixed and heated to 90° C. Thereto, 5.70 ml (150 mmol) of formic acid was added dropwise over 30 minutes, and heating was carried out at 100° C. for 12 hours. The resultant was cooled to room temperature, 2.000 g (50 mmol) of sodium hydroxide was added, and the mixture was stirred for 1 hour. Thereafter, suction filtration was carried out, and the filtrate was concentrated under reduced pressure. The obtained concentrate was distilled under reduced pressure (70 to 72° C./2 mmHg) to obtain the target 1,2,2,6,6-pentamethyl-4-piperidone (1-2) (actual yield: 13.532 g, percent yield: 80%).

### Synthesis of 7-aza-3.11-dithiadispiro[5.1.5.3]hexadecan-15-one (1-3)

Under an argon stream, 12.373 g (73.1 mmol) of 1,2,2,6,6-pentamethyl-4-piperidone (1-2) synthesized by the above-mentioned reaction and 25.000 g (215.2 mmol) of 4-oxothiane were dissolved in 100 ml of dimethyl sulfoxide (DMSO). Thereto, 23.001 g (430.0 mmol) of ammonium chloride and then 14 ml of a 40% benzyltrimethylammonium hydroxide (Triton B) aqueous solution were added, and the mixture was stirred at 50° C. for 10 hours.

Water was added to the reaction solution, the pH was adjusted to 1 by 5% hydrochloric acid, and washing was carried out with diethyl ether. The pH of the aqueous layer was adjusted to 9 by a 10% potassium carbonate aqueous solution, and extraction was carried out with ethyl acetate. The organic layer was washed with a saturated sodium chloride aqueous solution, and dried with magnesium sulfate. Subsequently, the resultant was concentrated under reduced pressure. Purification was carried out by silica gel column chromatography (hexane:ethyl acetate=3:1 to 1:1). Subsequently, reprecipitation was carried out with hexane-ethyl acetate to obtain the target 7-aza-3.11-dithiadispiro[5.1.5.3]hexadecan-15-one (1-3) (actual yield: 5.771 g, percent yield: 29%).

### Synthesis of 4-hydroxy-2,2,6,6-tetraethylpiperidine (1-4)

Under an argon stream, 320 ml of ethanol (EtOH) was added to 5.500 g (20.3 mmol) of 7-aza-3.11-dithiadispiro[5.1.5.3]hexadecan-15-one (1-3) synthesized by the above-mentioned reaction. Furthermore, 50.00 g of Raney-Ni (water suspension, Ni>92.5%, Al<6.5%) was added while washing with 60 ml of ethanol, and the mixture was stirred at 65° C. for 72 hours.

The reaction solution was filtered through Celite and the resultant was concentrated under reduced pressure. The pH of the concentrated solution was adjusted to 12 by a saturated potassium carbonate aqueous solution. Subsequently, extraction with ethyl acetate was carried out, and washing was carried out with a saturated sodium chloride aqueous solution. The resultant was concentrated under reduced pressure, 7% hydrochloric acid was added, and washing was carried out with diethyl ether. The pH was adjusted to 12 by a 5M potassium hydroxide aqueous solution. Subsequently, extraction with ethyl acetate was carried out, and washing was carried out with a saturated sodium chloride aqueous solution. After drying with magnesium sulfate, the resultant was concentrated under reduced pressure. Purification was carried out by silica gel column chromatography (hexane:ethyl acetate=5:1) to obtain the target 4-hydroxy-2,2,6,6-tetraethylpiperidine (1-4) (actual yield: 2.599 g, percent yield: 60%).

### Synthesis of 4-hydroxy-2,2,6,6-tetraethylpiperidin-1-oxyl (1-5)

Under an argon stream, 2.599 g (12.2 mmol) of 4-hydroxy-2,2,6,6-tetraethylpiperidine (1-4) synthesized by the above-mentioned reaction was dissolved in 400 ml of dichloromethane, and cooling was carried out in an ice bath. Thereto, 80 ml of a dichloromethane solution containing 24.4 mmol of meta-chloroperbenzoic acid (mCPBA) was added dropwise over 45 minutes, and the mixture was stirred at room temperature for 3 hours. The reaction solution was concentrated under reduced pressure, and the obtained crude product was dissolved in diethyl ether. Washing was carried out with a saturated sodium carbonate aqueous solution, then washing was carried out with a saturated sodium chloride aqueous solution, and thereafter, drying was carried out with magnesium sulfate. The resultant was concentrated under reduced pressure. Subsequently, purification was carried out by silica gel column chromatography (hexane:ethyl acetate=5:1) to obtain the target 4-hydroxy-2,2,6,6-tetraethylpiperidin-1-oxyl (1-5) (actual yield: 1.811 g, percent yield: 65%).

### Synthesis of 4-(3-((1,1,1,3,3,3-hexafluoro-2-(trifluoromethyl)propan-2-yl)oxy)-2,2-bis(((1,1,1,3,3,3-hexafluoro-2-(trifluoromethyl)propan-2-yl)oxy)methyl)propoxy)-2,2,6,6-tetraethylpiperidin-1-oxyl (12)

Under an argon stream, 5 ml of dimethylformamide was added to 0.349 g (8.00 mmol) of 55% sodium hydride, and the mixture was stirred at room temperature for 10 minutes. Thereto, 20 ml of a dimethylformamide solution containing 1.811 g (7.93 mmol) of 4-hydroxy-2,2,6,6-tetraethylpiperidin-1-oxyl (1-5) synthesized by the above-mentioned reaction was added dropwise over 10 minutes, and the mixture was stirred at room temperature for 3 hours. Furthermore, 5.972 g (7.00 mmol) of 2-(3-bromo-2,2-bis(((1,1,1,3,3,3-hexafluoro-2-(trifluoromethyl)propan-2-yl)oxy)methyl)propoxy)-1,1,1,3,3,3-hexafluoro-2-(trifluoromethyl)propane (1-1) synthesized by the above-mentioned reaction was added in an ice bath, and the mixture was stirred at 60° C. for 15 hours.

Water was added to the reaction solution, and extraction was carried out with ethyl acetate. Thereafter, washing was carried out with water, and the organic layer was dried with magnesium sulfate. The resultant was concentrated under reduced pressure. Subsequently, purification was carried out by silica gel column chromatography (hexane:ethyl acetate=95:5) to obtain the target 4-(3-((1,1,1,3,3,3-hexafluoro-2-(trifluoromethyl)propan-2-yl)oxy)-2,2-bis(((1,1,1,3,3,3-hexafluoro-2-(trifluoromethyl)propan-2-yl)oxy)methyl)propoxy)-2,2,6,6-tetraethylpiperidin-1-oxyl (12) (actual yield: 2.101 g, percent yield: 30%).

When the obtained compound was subjected to mass spectrometry, a peak was confirmed at m/z=1000 (M′). From this result, it was confirmed that the synthesized compound was the compound represented by Formula (12). Furthermore, the degree of purity of the compound represented by Formula (12) confirmed by high-performance liquid chromatography (HPLC) was 98.9%.

### Example 3

### (Synthesis of Compound 13)

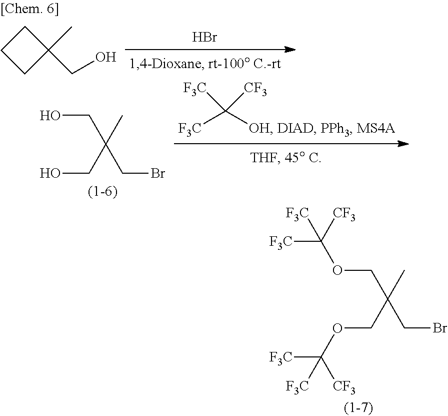

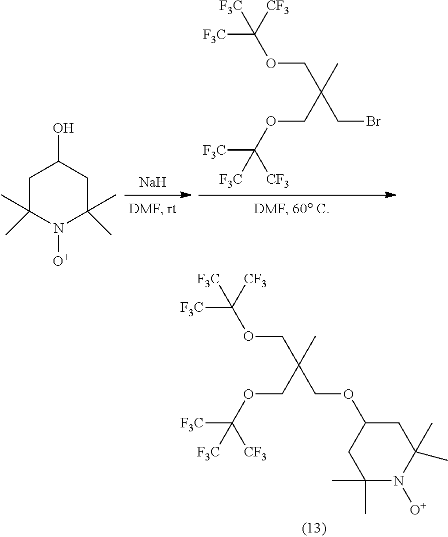

### Synthesis of 2-(bromomethyl)-2-methyl-1,3-propanediol (1-6)

5.450 g (53.4 mmol) of 3-methyl-3-octanemethanol was dissolved in 25 ml of 1,4-dioxane, and 7.20 nil (64.0 mmol) of a 48% hydrogen bromide aqueous solution was added dropwise over 10 minutes, and the mixture was stirred at 100° C. for 4 hours. Thereafter, the reaction solution was cooled to room temperature and the resultant was concentrated under reduced pressure. The concentrate was dissolved in diethyl ether and washed with a saturated sodium chloride aqueous solution and then the concentrate was washed with a sodium carbonate aqueous solution. Drying was carried out with magnesium sulfate. Subsequently, the resultant was concentrated under reduced pressure to obtain the target 2-(bromomethyl)-2-methyl-1,3-propanediol (1-6) (actual yield: 8.993 g, percent yield: 92%).

### Synthesis of 2-(3-bromo-2-(((1,1,1,3,3,3-hexafluoro-2-(trifluoromethyl)propan-2-yl)oxy)methyl)-2-methylpropoxy)-1,1,1,3,3,3-hexafluoro-2-(trifluoromethyl)propane (1-7)

Under an argon stream, 7.761 g (42.4 mmol) of 2-(bromomethyl)-2-methyl-1,3-propanediol (1-6) synthesized by the above-mentioned reaction, 27.777 g (105.9 mmol) of triphenylphosphine (PPh₃), 10.000 g of Molecular Sieve (MS) 4A, and 130 ml of tetrahydrofuran (THF) were mixed and cooled in an ice bath. Thereto, 20.6 ml (105.9 mmol) of diisopropylazodicarboxylic acid ester (DIAD) was added dropwise over 10 minutes, and the mixture was stirred for 20 minutes. Furthermore, 25.000 g (105.9 mmol) of nonafluoro-tert-butanol was added at one time, and the mixture was stirred at 45° C. for 72 hours.

The reaction solution was filtered. Subsequently, the resultant was concentrated under reduced pressure, and purification was carried out by silica gel column chromatography (hexane:ethyl acetate=9:1) to obtain the target compound 2-(3-bromo-2-(((1,1,1,3,3,3-hexafluoro-2-(trifluoromethyl)propan-2-yl)oxy)methyl)-2-methylpropoxy)-1,1,1,3,3,3-hexafluoro-2-(trifluoromethyl)propane (1-7) (actual yield: 21.003 g, percent yield: 80%).

### Synthesis of 4-(3-((1,1,1,3,3,3-hexafluoro-2-(trifluoromethyl)propan-2-yl)oxy)-2-(((1,1,1,3,3,3-hexafluoro-2-(trifluoromethyl)propan-2-yl)oxy)methyl)-2-methylpropoxy)-2,2,6,6-tetramethylpiperidin-1-oxyl (13)

Under an argon stream, 10 ml of dimethylformamide (DMF) was added to 0.567 g (13.0 mmol) of 55% sodium hydride, and the mixture was stirred at room temperature for 10 minutes. Thereto, 20 ml of a dimethylformamide solution containing 2.239 g (13.0 mmol) of 4-hydroxy-2,2,6,6-tetramethylpiperidin-1-oxyl was added dropwise over 10 minutes, and the mixture was stirred at room temperature for 3 hours. Furthermore, 5.993 g (9.68 mmol) of 2-(3-bromo-2-(((1,1,1,3,3,3-hexafluoro-2-(trifluoromethyl)propan-2-yl)oxy)methyl)-2-methylpropoxy)-1,1,1,3,3,3-hexafluoro-2-(trifluoromethyl)propane (1-7) synthesized by the above-mentioned reaction and 60 ml of dimethylformamide were added in an ice bath, and the mixture was stirred at 60° C. for 16 hours.

Water was added to the reaction solution, and extraction was carried out with ethyl acetate. Thereafter, washing was carried out with a saturated sodium chloride aqueous solution, and the organic layer was dried with magnesium sulfate. The resultant was concentrated under reduced pressure. Subsequently, purification was carried out by silica gel column chromatography (hexane:ethyl acetate=9:1 to 4:1) to obtain the target 4-(3-((1,1,1,3,3,3-hexafluoro-2-(trifluoromethyl)propan-2-yl)oxy)-2-(((1,1,1,3,3,3-hexafluoro-2-(trifluoromethyl)propan-2-yl)oxy)methyl)-2-methylpropoxy)-2,2,6,6-tetramethylpiperidin-1-oxyl (13) (actual yield: 5.364 g, percent yield: 78%).

When the obtained compound was subjected to mass spectrometry, a peak was confirmed at m/z=701 (M⁺). From this result, it was confirmed that the synthesized compound was the compound represented by Formula (13). Furthermore, the degree of purity of the compound represented by Formula (13) confirmed by high-performance liquid chromatography (HPLC) was 98.8%.

### Example 4

### (Synthesis of Compound 14)

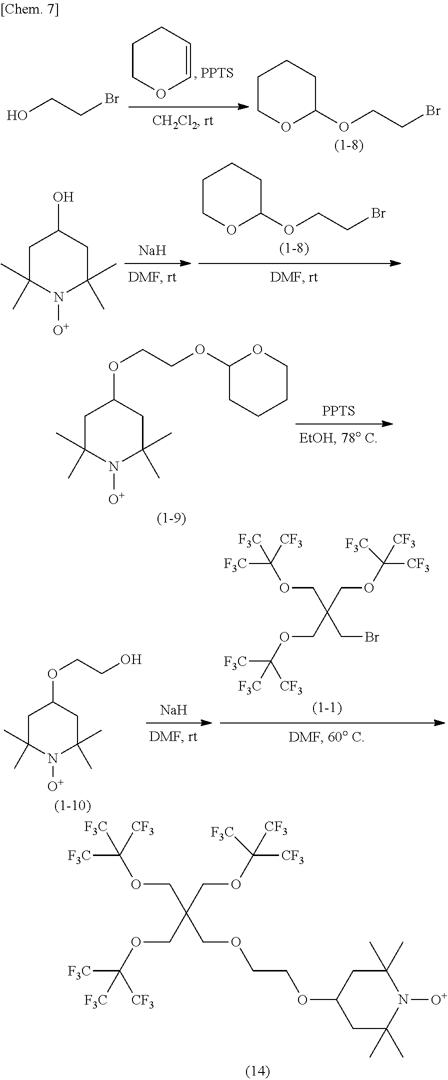

### Synthesis of 2-(2-bromoethoxy)tetrahydro-2H-pyran (1-8)

Under an argon stream, 3.749 g (30.0 mmol) of 2-bromo-1-ethanol was dissolved in 150 ml of dichloromethane, 3.30 ml (36.0 mmol) of 3,4-dihydro-2H-pyrane was added, and then 1.508 g (6.00 mmol) of pyridinium p-toluenesulfonate (PPTS) was added, and the mixture was stirred at room temperature for 18 hours. The reaction solution was concentrated under reduced pressure, and then purification was carried out by silica gel column chromatography (hexane:ethyl acetate=9:1) to obtain the target 2-(2-bromoethoxy)tetrahydro-2H-pyran (1-8) (actual yield: 5.206 g, percent yield: 83%).

### Synthesis of 2,2,6,6-tetramethyl-4-(2-((tetrahydro-2H-pyran-2-yl)oxy)ethoxy)piperidin-1-oxyl (1-9)

Under an argon stream, 10 ml of dimethylformamide (DMF) was added to 0.524 g (12.0 mmol) of 55% sodium hydride, and the mixture was stirred at room temperature for 10 minutes. Thereto, 20 ml of a dimethylformamide solution containing 1.723 g (10.0 mmol) of 4-hydroxy-2,2,6,6-tetramethylpiperidin-1-oxyl was added dropwise over 10 minutes, and the mixture was stirred at room temperature for 3 hours. Furthermore, 10 ml of a dimethylformamide solution containing 2.927 g (14.0 mmol) of 2-(2-bromoethoxy)tetrahydro-2H-pyran (1-8) synthesized by the above-mentioned reaction was added in an ice bath, and the mixture was stirred at room temperature for 15 hours.

Water was added to the reaction solution, and extraction was carried out with diethyl ether. Thereafter, washing was carried out with water, and the organic layer was dried with magnesium sulfate. The resultant was concentrated under reduced pressure. Subsequently, purification was carried out by silica gel column chromatography (hexane:ethyl acetate=9:1) to obtain the target 2,2,6,6-tetramethyl-4-(2-((tetrahydro-2H-pyran-2-yl)oxy)ethoxy)piperidin-1-oxyl (1-9) (actual yield: 1.382 g, percent yield: 46%).

### Synthesis of 4-(2-hydroxyethoxy)-2,2,6,6-tetramethylpiperidin-1-oxyl (1-10)

1.382 g (4.60 mmol) of 2,2,6,6-tetramethyl-4-(2-((tetrahydro-2H-pyran-2-yl)oxy)ethoxy)piperidin-1-oxyl (1-9) synthesized by the above-mentioned reaction, and 0.116 g (0.46 mmol) of pyridinium p-toluenesulfonate (PPTS) were dissolved in 200 ml of ethanol (EtOH), and the mixture was stirred at 78° C. for 3 hours. The reaction solution was concentrated under reduced pressure. Subsequently, purification was carried out by silica gel column chromatography (hexane:ethyl acetate=4:1) to obtain the target 4-(2-hydroxyethoxy)-2,2,6,6-tetramethylpiperidin-1-oxyl (1-10) (actual yield: 0.895 g, percent yield: 90%).

### <Synthesis of Compound (14)>

Under an argon stream, 5 ml of dimethylformamide (DMF) was added to 0.218 g (5.00 mmol) of 55% sodium hydride, and the mixture was stirred at room temperature for 10 minutes. Thereto, 15 ml of a dimethylformamide solution containing 0.895 g (4.14 mmol) of 4-(2-hydroxyethoxy)-2,2,6,6-tetramethylpiperidin-1-oxyl (1-10) synthesized by the above-mentioned reaction was added dropwise over 10 minutes, and the mixture was stirred at room temperature for 3 hours. Furthermore, 15 ml of a dimethylformamide solution containing 2.943 g (3.45 mmol) of 2-(3-bromo-2,2-bis(((1,1,1,3,3,3-hexafluoro-2-(trifluoromethyl)propan-2-yl)oxy)methyl)propoxy)-1,1,1,3,3,3-hexafluoro-2-(trifluoromethyl)propane (1-1) synthesized by the above-mentioned reaction was added in an ice bath, and the mixture was stirred at 60° C. for 15 hours.

Water was added to the reaction solution, and extraction was carried out with ethyl acetate. Thereafter, washing was carried out with a saturated sodium chloride aqueous solution, and the organic layer was dried with magnesium sulfate. The resultant was concentrated under reduced pressure. Subsequently, purification was carried out by silica gel column chromatography (hexane:ethyl acetate=9:1) to obtain the target compound (14) (actual yield: 2.046 g, percent yield: 60%).

When the obtained compound was subjected to mass spectrometry, a peak was confirmed at m/z=988 (M⁺). From this result, it was confirmed that the synthesized compound was the compound represented by Formula (14). Furthermore, the degree of purity of the compound represented by Formula (14) confirmed by high-performance liquid chromatography (HPLC) was 98.7%.

### Example 5

### (Synthesis of Compound 15)

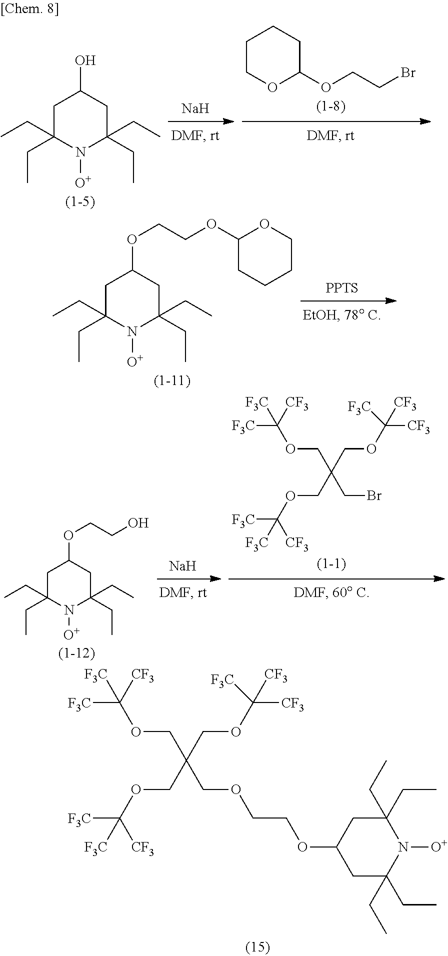

### Synthesis of 2,2,6,6-tetraethyl-4-(2-((tetrahydro-2H-pyran-2-yl)oxy)ethoxy)piperidin-1-oxyl (1-11)

Under an argon stream, 10 ml of dimethylformamide (DMF) was added to 0.524 g (12.0 mmol) of 55% sodium hydride, and the mixture was stirred at room temperature for 10 minutes. Thereto, 20 ml of a dimethylformamide solution containing 2.284 g (10.0 mmol) of 4-hydroxy-2,2,6,6-tetraethylpiperidin-1-oxyl (1-5) synthesized by the above-mentioned reaction was added dropwise over 10 minutes, and the mixture was stirred at room temperature for 3 hours. Furthermore, 10 ml of a dimethylformamide solution containing 2.927 g (14.0 mmol) of 2-(2-bromoethoxy)tetrahydro-2H-pyran (1-8) synthesized by the above-mentioned reaction was added in an ice bath, and the mixture was stirred at room temperature for 15 hours.

Water was added to the reaction solution, and extraction was carried out with diethyl ether. Thereafter, washing was carried out with water, and the organic layer was dried with magnesium sulfate. The resultant was concentrated under reduced pressure. Subsequently, purification was carried out by silica gel column chromatography (hexane:ethyl acetate=9:1) to obtain the target 2,2,6,6-tetraethyl-4-(2-((tetrahydro-2H-pyran-2-yl)oxy)ethoxy)piperidin-1-oxyl (1-11) (actual yield: 1.426 g, percent yield: 40%).

### Synthesis of 4-(2-hydroxyethoxy)-2,2,6,6-tetraethylpiperidin-1-oxyl (1-12)

1.426 g (4.00 mmol) of 2,2,6,6-tetraethyl-4-(2-((tetrahydro-2H-pyran-2-yl)oxy)ethoxy)piperidin-1-oxyl (1-11) synthesized by the above-mentioned reaction, and 0.100 g (0.40 mmol) of pyridinium p-toluenesulfonate (PPTS) were dissolved in 200 ml of ethanol (EtOH), and the mixture was stirred at 78° C. for 3 hours. The reaction solution was concentrated under reduced pressure. Subsequently, purification was carried out by silica gel column chromatography (hexane:ethyl acetate=4:1) to obtain the target 4-(2-hydroxyethoxy)-2,2,6,6-tetraethylpiperidin-1-oxyl (1-12) (actual yield: 1.002 g, percent yield: 92%).

### <Synthesis of Compound (15)>

Under an argon stream, 5 ml of dimethylformamide (DMF) was added to 0.193 g (4.42 mmol) of 55% sodium hydride, and the mixture was stirred at room temperature for 10 minutes. Thereto, 15 ml of a dimethylformamide solution containing 1.002 g (3.68 mmol) of 4-(2-hydroxyethoxy)-2,2,6,6-tetraethylpiperidin-1-oxyl (1-12) synthesized by the above-mentioned reaction was added dropwise over 10 minutes, and the mixture was stirred at room temperature for 3 hours. Furthermore, 15 ml of a dimethylformamide solution containing 2.619 g (3.07 mmol) of 2-(3-bromo-2,2-bis(((1,1,1,3,3,3-hexafluoro-2-(trifluoromethyl)propan-2-yl)oxy)methyl)propoxy)-1,1,1,3,3,3-hexafluoro-2-(trifluoromethyl)propane (1-1) synthesized by the above-mentioned reaction was added in an ice bath, and the mixture was stirred at 60° C. for 18 hours.

Water was added to the reaction solution, and extraction was carried out with ethyl acetate. Thereafter, washing was carried out with a saturated sodium chloride aqueous solution, and the organic layer was dried with magnesium sulfate. The resultant was concentrated under reduced pressure. Subsequently, purification was carried out by silica gel column chromatography (hexane:ethyl acetate=9:1) to obtain the target compound (15) (actual yield: 1.860 g, percent yield: 58%).

When the obtained compound was subjected to mass spectrometry, a peak was confirmed at m/z=1044 (Me). From this result, it was confirmed that the synthesized compound was the compound represented by Formula (15). Furthermore, the degree of purity of the compound represented by Formula (15) confirmed by high-performance liquid chromatography (HPLC) was 98.3%.

### Example 6

### (Synthesis of Compound 16)

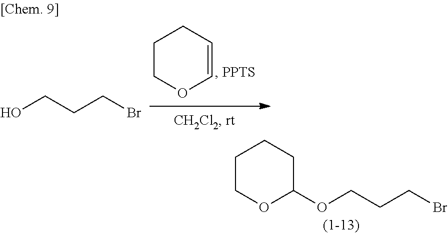

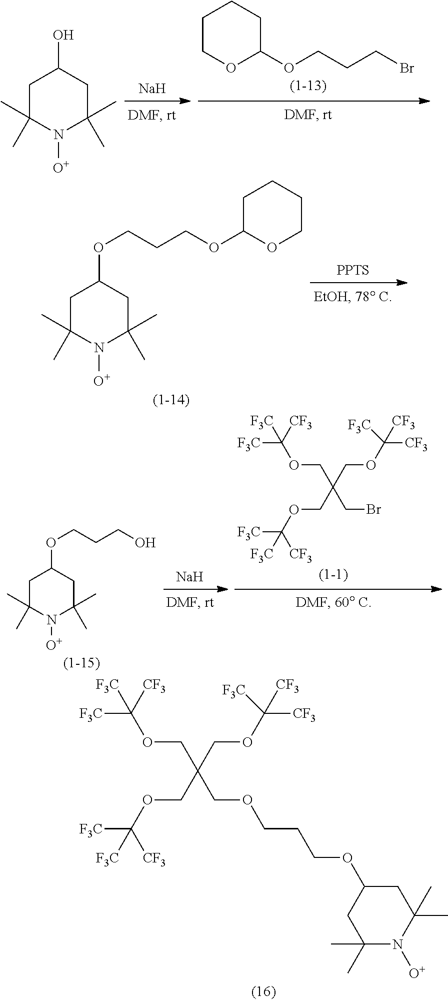

### Synthesis of 2-(3-bromopropoxy)tetrahydro-2H-pyran (1-13)

Under an argon stream, 4.170 g (30.0 mmol) of 3-bromo-1-propanol was dissolved in 150 ml of dichloromethane, 3.30 ml (36.0 mmol) of 3,4-dihydro-2H-pyrane was added, and then 1.508 g (6.00 mmol) of pyridinium p-toluenesulfonate (PPTS) was added, and the mixture was stirred at room temperature for 18 hours. The reaction solution was concentrated under reduced pressure. Subsequently, purification was carried out by silica gel column chromatography (hexane:ethyl acetate=9:1) to obtain the target 2-(3-bromopropoxy)tetrahydro-2H-pyran (1-13) (actual yield: 5.355 g, percent yield: 80%).

### Synthesis of 2,2,6,6-tetramethyl-4-(3-((tetrahydro-2H-pyran-2-yl)oxy)propoxy)piperidin-1-oxyl (1-14)

Under an argon stream, 10 ml of dimethylformamide (DMF) was added to 0.524 g (12.0 mmol) of 55% sodium hydride, and the mixture was stirred at room temperature for 10 minutes. Thereto, 20 ml of a dimethylformamide solution containing 1.723 g (10.0 mmol) of 4-hydroxy-2,2,6,6-tetramethylpiperidin-1-oxyl was added dropwise over 10 minutes, and the mixture was stirred at room temperature for 3 hours. Furthermore, 10 ml of a dimethylformamide solution containing 3.124 g (14.0 mmol) of 2-(3-bromopropoxy)tetrahydro-2H-pyran (1-13) synthesized by the above-mentioned reaction was added in an ice bath, and the mixture was stirred at room temperature for 15 hours.

Water was added to the reaction solution, and extraction was carried out with diethyl ether. Thereafter, washing was carried out with water, and the organic layer was dried with magnesium sulfate. The resultant was concentrated under reduced pressure. Subsequently, purification was carried out by silica gel column chromatography (hexane:ethyl acetate=9:1) to obtain the target 2,2,6,6-tetramethyl-4-(3-((tetrahydro-2H-pyran-2-yl)oxy)propoxy)piperidin-1-oxyl (1-14) (actual yield: 1.572 g, percent yield: 50%).

### Synthesis of 4-(3-hydroxypropoxy)-2,2,6,6-tetramethylpiperidin-1-oxyl (1-15)

1.572 g (5.00 mmol) of 2,2,6,6-tetramethyl-4-(3-((tetrahydro-2H-pyran-2-yl)oxy)propoxy)piperidin-1-oxyl (1-14) synthesized by the above-mentioned reaction, and 0.126 g (0.50 mmol) of pyridinium p-toluenesulfonate (PPTS) were dissolved in 200 ml of ethanol (EtOH), and the mixture was stirred at 78° C. for 3 hours. The reaction solution was concentrated under reduced pressure. Subsequently, purification was carried out by silica gel column chromatography (hexane:ethyl acetate=4:1) to obtain the target 4-(3-hydroxypropoxy)-2,2,6,6-tetramethylpiperidin-1-oxyl (1-15) (actual yield: 0.990 g, percent yield: 86%).

### <Synthesis of Compound (16)>

Under an argon stream, 5 ml of dimethylformamide was added to 0.225 g (5.16 mmol) of 55% sodium hydride, and the mixture was stirred at room temperature for 10 minutes. Thereto, 15 ml of a dimethylformamide solution containing 0.990 g (4.30 mmol) of 4-(3-hydroxypropoxy)-2,2,6,6-tetramethylpiperidin-1-oxyl (1-15) synthesized by the above-mentioned reaction was added dropwise over 10 minutes, and the mixture was stirred at room temperature for 3 hours. Furthermore, 15 ml of a dimethylformamide solution containing 3.057 g (3.58 mmol) of 2-(3-bromo-2,2-bis(((1,1,1,3,3,3-hexafluoro-2-(trifluoromethyl)propan-2-yl)oxy)methyl)propoxy)-1,1,1,3,3,3-hexafluoro-2-(trifluoromethyl)propane (1-1) synthesized by the above-mentioned reaction was added in an ice bath, and the mixture was stirred at 60° C. for 13 hours.

Water was added to the reaction solution, and extraction was carried out with ethyl acetate. Thereafter, washing was carried out with a saturated sodium chloride aqueous solution, and the organic layer was dried with magnesium sulfate. The resultant was concentrated under reduced pressure. Subsequently, purification was carried out by silica gel column chromatography (hexane:ethyl acetate=9:1) to obtain the target compound (16) (actual yield: 2.716 g, percent yield: 63%).

When the obtained compound was subjected to mass spectrometry, a peak was confirmed at m/z=1002 (M). From this result, it was confirmed that the synthesized compound was the compound represented by Formula (16). Furthermore, the degree of purity of the compound represented by Formula (16) confirmed by high-performance liquid chromatography (HPLC) was 98.8%.

### Example 7

### (Synthesis of Compound 17)

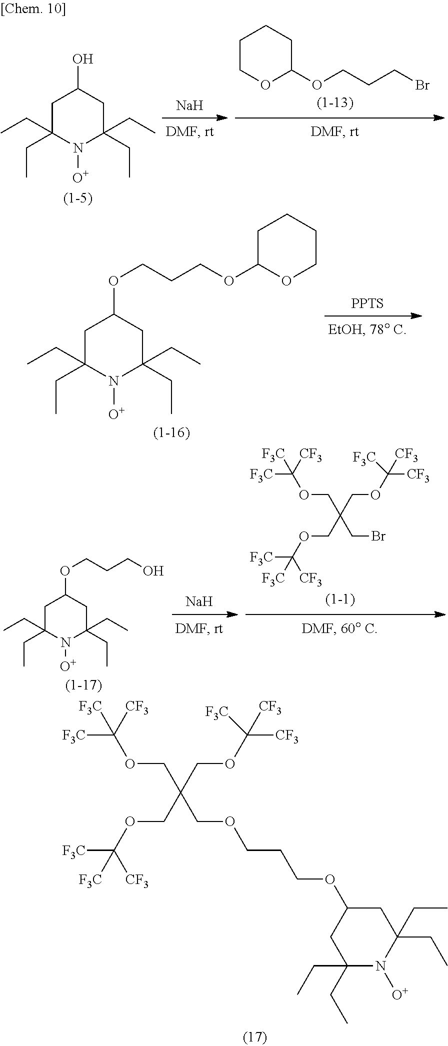

### Synthesis of 2,2,6,6-tetraethyl-4-(3-((tetrahydro-2H-pyran-2-yl)oxy)propoxy)piperidin-1-oxyl (1-16)

Under an argon stream, 10 ml of dimethylformamide (DMF) was added to 0.524 g (12.0 mmol) of 55% sodium hydride, and the mixture was stirred at room temperature for 10 minutes. Thereto, 20 ml of a dimethylformamide solution containing 2.284 g (10.0 mmol) of 4-hydroxy-2,2,6,6-tetraethylpiperidin-1-oxyl (1-5) synthesized by the above-mentioned reaction was added dropwise over 10 minutes, and the mixture was stirred at room temperature for 3 hours. Furthermore, 10 ml of a dimethylformamide solution containing 3.124 g (14.0 mmol) of 2-(3-bromopropoxy)tetrahydro-2H-pyran (1-13) synthesized by the above-mentioned reaction was added in an ice bath, and the mixture was stirred at room temperature for 15 hours.

Water was added to the reaction solution, and extraction was carried out with diethyl ether. Thereafter, washing was carried out with water, and the organic layer was dried with magnesium sulfate. The resultant was concentrated under reduced pressure. Subsequently, purification was carried out by silica gel column chromatography (hexane:ethyl acetate=9:1) to obtain the target 2,2,6,6-tetraethyl-4-(3-((tetrahydro-2H-pyran-2-yl)oxy)propoxy)piperidin-1-oxyl (1-16) (actual yield: 1.964 g, percent yield: 53%).

### Synthesis of 4-(3-hydroxypropoxy)-2,2,6,6-tetraethylpiperidin-1-oxyl (1-17)

1.964 g (5.30 mmol) of 2,2,6,6-tetraethyl-4-(3-((tetrahydro-2H-pyran-2-yl)oxy)propoxy)piperidin-1-oxyl (1-16) synthesized by the above-mentioned reaction, and 0.133 g (0.53 mmol) of pyridinium p-toluenesulfonate (PPTS) were dissolved in 200 ml of ethanol (EtOH), and the mixture was stirred at 78° C. for 3 hours. The reaction solution was concentrated under reduced pressure. Subsequently, purification was carried out by silica gel column chromatography (hexane:ethyl acetate=4:1) to obtain the target 4-(3-hydroxypropoxy)-2,2,6,6-tetraethylpiperidin-1-oxyl (1-17) (actual yield: 1.230 g, percent yield: 81%).

### <Synthesis of Compound (17)>

Under an argon stream, 5 ml of dimethylformamide was added to 0.225 g (5.16 mmol) of 55% sodium hydride, and the mixture was stirred at room temperature for 10 minutes. Thereto, 15 ml of a dimethylformamide solution containing 1.230 g (4.29 mmol) of 4-(3-hydroxypropoxy)-2,2,6,6-tetraethylpiperidin-1-oxyl (1-17) synthesized by the above-mentioned reaction was added dropwise over 10 minutes, and the mixture was stirred at room temperature for 3 hours. Furthermore, 15 ml of a dimethylformamide solution containing 3.057 g (3.58 mmol) of 2-(3-bromo-2,2-bis(((1,1,1,3,3,3-hexafluoro-2-(trifluoromethyl)propan-2-yl)oxy)methyl)propoxy)-1,1,1,3,3,3-hexafluoro-2-(trifluoromethyl)propane (1-1) synthesized by the above-mentioned reaction was added in an ice bath, and the mixture was stirred at 60° C. for 13 hours.

Water was added to the reaction solution, and extraction was carried out with ethyl acetate. Thereafter, washing was carried out with a saturated sodium chloride aqueous solution, and the organic layer was dried with magnesium sulfate. The resultant was concentrated under reduced pressure. Subsequently, purification was carried out by silica gel column chromatography (hexane:ethyl acetate=9:1) to obtain the target compound (17) (actual yield: 1.971 g, percent yield: 52%).

When the obtained compound was subjected to mass spectrometry, a peak was confirmed at m/z=1058 (M⁺). From this result, it was confirmed that the synthesized compound was the compound represented by Formula (17). Furthermore, the degree of purity of the compound represented by Formula (17) confirmed by high-performance liquid chromatography (HPLC) was 98.0%.

### Example 8

### (Synthesis of Compound 18)

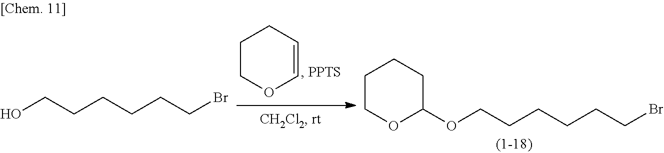

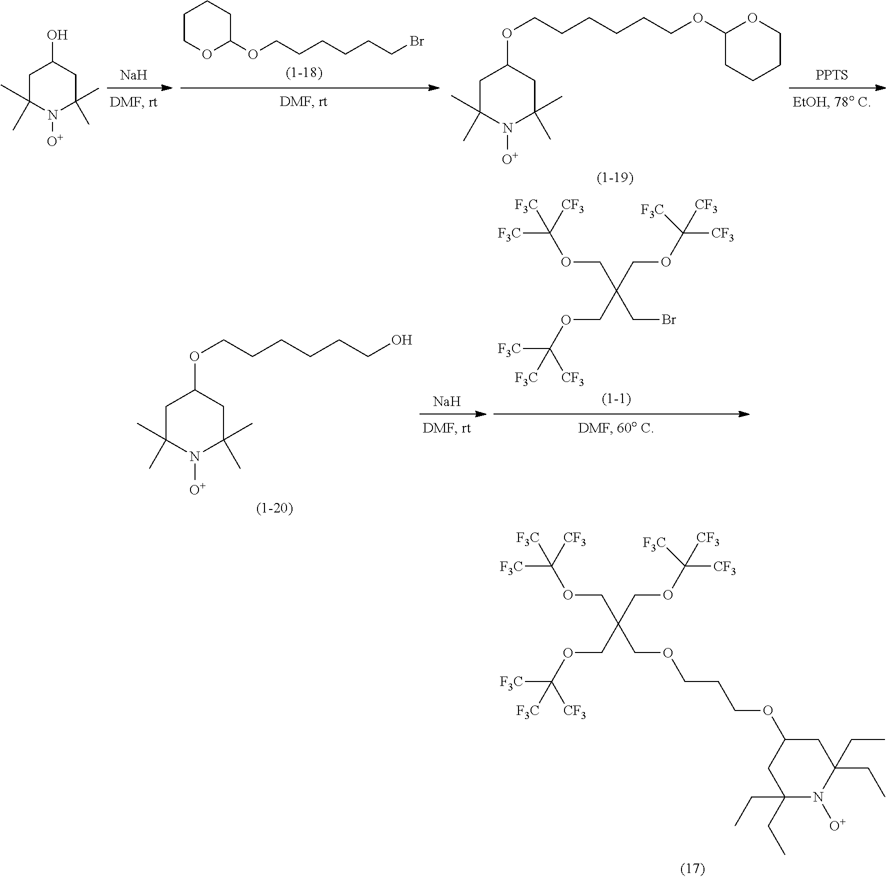

### Synthesis of 2-((6-bromohexyl)oxy)tetrahydro-2H-pyran (1-18)

Under an argon stream, 5.432 g (30.0 mmol) of 6-bromo-1-hexanol was dissolved in 150 ml of dichloromethane, 3.30 ml (36.0 mmol) of 3,4-dihydro-2H-pyrane was added, and then 1.508 g (6.00 mmol) of pyridinium p-toluenesulfonate (PPTS) was added, and the mixture was stirred at room temperature for 18 hours. The reaction solution was concentrated under reduced pressure. Subsequently, purification was carried out by silica gel column chromatography (hexane:ethyl acetate=9:1) to obtain the target 2-((6-bromohexyl)oxy)tetrahydro-2H-pyran (1-18) (actual yield: 6.762 g, percent yield: 85%).

### Synthesis of 2,2,6,6-tetramethyl-4-((6-((tetrahydro-2H-pyran-2-yl)oxy)hexyl)oxy)piperidin-1-oxyl (1-19)

Under an argon stream, 10 ml of dimethylformamide (DMF) was added to 0.524 g (12.0 mmol) of 55% sodium hydride, and the mixture was stirred at room temperature for 10 minutes. Thereto, 20 ml of a dimethylformamide solution containing 1.723 g (10.0 mmol) of 4-hydroxy-2,2,6,6-tetramethylpiperidin-1-oxyl was added dropwise over 10 minutes, and the mixture was stirred at room temperature for 3 hours. Furthermore, 10 ml of a dimethylformamide solution containing 3.713 g (14.0 mmol) of 2-((6-bromohexyl)oxy)tetrahydro-2H-pyran (1-18) synthesized by the above-mentioned reaction was added in an ice bath, and the mixture was stirred at room temperature for 18 hours.

Water was added to the reaction solution, and extraction was carried out with diethyl ether. Thereafter, washing was carried out with water, and the organic layer was dried with magnesium sulfate. The resultant was concentrated under reduced pressure. Subsequently, purification was carried out by silica gel column chromatography (hexane:ethyl acetate=9:1) to obtain the target 2,2,6,6-tetramethyl-4-((6-((tetrahydro-2H-pyran-2-yl)oxy)hexyl)oxy)piperidin-1-oxyl (1-19) (actual yield: 1.426 g, percent yield: 40%).

### Synthesis of 4-((6-hydroxyhexyl)oxy)-2,2,6,6-tetramethylpiperidin-1-oxyl (1-20)

1.426 g (4.00 mmol) of 2,2,6,6-tetramethyl-4-((6-((tetrahydro-2H-pyran-2-yl)oxy)hexyl)oxy)piperidin-1-oxyl (1-19) synthesized by the above-mentioned reaction, and 0.101 g (0.40 mmol) of pyridinium p-toluenesulfonate (PPTS) were dissolved in 200 ml of ethanol (EtOH), and the mixture was stirred at 78° C. for 3 hours.

The reaction solution was concentrated under reduced pressure. Subsequently, purification was carried out by silica gel column chromatography (hexane:ethyl acetate=4:1) to obtain the target 4-((6-hydroxyhexyl)oxy)-2,2,6,6-tetramethylpiperidin-1-oxyl (1-20) (actual yield: 0.959 g, percent yield: 88%).

### <Synthesis of Compound (18)>

Under an argon stream, 5 ml of dimethylformamide (DMF) was added to 0.184 g (4.22 mmol) of 55% sodium hydride, and the mixture was stirred at room temperature for 10 minutes. Thereto, 15 ml of a dimethylformamide solution containing 0.959 g (3.52 mmol) of 4-((6-hydroxyhexyl)oxy)-2,2,6,6-tetramethylpiperidin-1-oxyl (1-20) synthesized by the above-mentioned reaction was added dropwise over 10 minutes, and the mixture was stirred at room temperature for 3 hours. Furthermore, 15 ml of a dimethylformamide solution containing 2.500 g (2.93 mmol) of 2-(3-bromo-2,2-bis(((1,1,1,3,3,3-hexafluoro-2-(trifluoromethyl)propan-2-yl)oxy)methyl)propoxy)-1,1,1,3,3,3-hexafluoro-2-(trifluoromethyl)propane (1-1) synthesized by the above-mentioned reaction was added in an ice bath, and the mixture was stirred at 60° C. for 13 hours.

Water was added to the reaction solution, and extraction was carried out with ethyl acetate. Thereafter, washing was carried out with a saturated sodium chloride aqueous solution, and the organic layer was dried with magnesium sulfate. The resultant was concentrated under reduced pressure. Subsequently, purification was carried out by silica gel column chromatography (hexane:ethyl acetate=9:1) to obtain the target compound (18) (actual yield: 1.653 g, percent yield: 54%).

When the obtained compound was subjected to mass spectrometry, a peak was confirmed at m/z=1044 (M′). From this result, it was confirmed that the synthesized compound was the compound represented by Formula (18). Furthermore, the degree of purity of the compound represented by Formula (18) confirmed by high-performance liquid chromatography (HPLC) was 98.8%.

### Example 9

### (Synthesis of Compound 19)

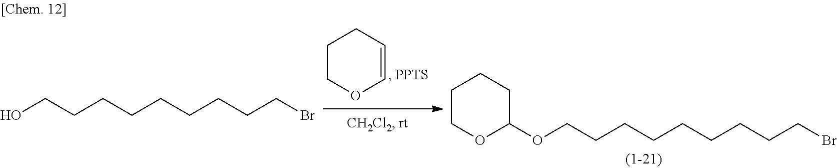

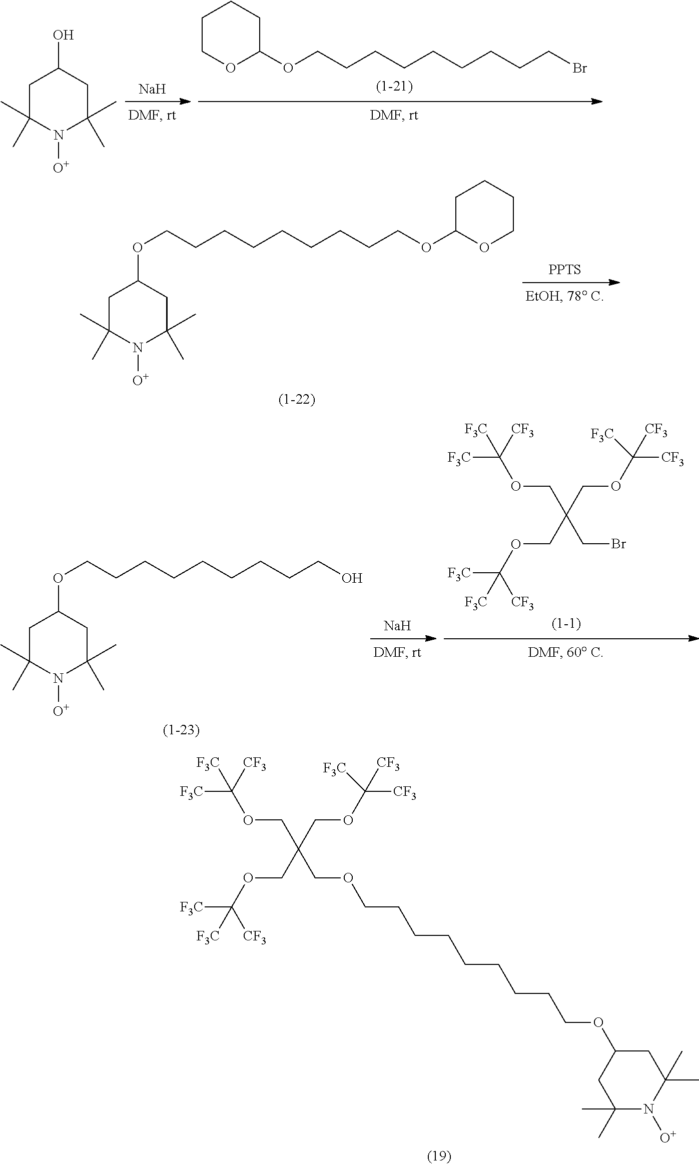

### Synthesis of 2-((9-bromononyl)oxy)tetrahydro-2H-pyran (1-21)

Under an argon stream, 6.695 g (30.0 mmol) of 9-bromo-1-nonanol was dissolved in 150 ml of dichloromethane, 3.30 ml (36.0 mmol) of 3,4-dihydro-2H-pyrane was added, and then 1.508 g (6.00 mmol) of pyridinium p-toluenesulfonate (PPTS) was added, and the mixture was stirred at room temperature for 18 hours. The reaction solution was concentrated under reduced pressure. Subsequently, purification was carried out by silica gel column chromatography (hexane:ethyl acetate=9:1) to obtain the target 2-((9-bromononyl)oxy)tetrahydro-2H-pyran (1-21) (actual yield: 7.374 g, percent yield: 80%).

### Synthesis of 2,2,6,6-tetramethyl-4-((9-((tetrahydro-2H-pyran-2-yl)oxy)nonyl)oxy)piperidin-1-oxyl (1-22)

Under an argon stream, 10 ml of dimethylformamide (DMF) was added to 0.524 g (12.0 mmol) of 55% sodium hydride, and the mixture was stirred at room temperature for 10 minutes. Thereto, 20 ml of a dimethylformamide solution containing 1.723 g (10.0 mmol) of 4-hydroxy-2,2,6,6-tetramethylpiperidin-1-oxyl was added dropwise over 10 minutes, and the mixture was stirred at room temperature for 3 hours. Furthermore, 20 ml of a dimethylformamide solution containing 4.302 g (14.0 mmol) of 2-((9-bromononyl)oxy)tetrahydro-2H-pyran (1-21) synthesized by the above-mentioned reaction was added in an ice bath, and the mixture was stirred at room temperature for 18 hours.

Water was added to the reaction solution, and extraction was carried out with diethyl ether. Thereafter, washing was carried out with water, and the organic layer was dried with magnesium sulfate. The resultant was concentrated under reduced pressure. Subsequently, purification was carried out by silica gel column chromatography (hexane:ethyl acetate=9:1) to obtain the target 2,2,6,6-tetramethyl-4-((9-((tetrahydro-2H-pyran-2-yl)oxy)nonyl)oxy)piperidin-1-oxyl (1-22) (actual yield: 1.714 g, percent yield: 43%).

### Synthesis of 4-((9-hydroxynonyl)oxy)-2,2,6,6-tetramethylpiperidin-1-oxyl (1-23)

1.714 g (4.30 mmol) of 2,2,6,6-tetramethyl-4-((9-((tetrahydro-2H-pyran-2-yl)oxy)nonyl)oxy)piperidin-1-oxyl (1-22) synthesized by the above-mentioned reaction, and 0.108 g (0.43 mmol) of pyridinium p-toluenesulfonate (PPTS) were dissolved in 200 ml of ethanol (EtOH), and the mixture was stirred at 78° C. for 3 hours. The reaction solution was concentrated under reduced pressure. Subsequently, purification was carried out by silica gel column chromatography (hexane:ethyl acetate=4:1) to obtain the target 4-((9-hydroxynonyl)oxy)-2,2,6,6-tetramethylpiperidin-1-oxyl (1-23) (actual yield: 1.190 g, percent yield: 88%).

### <Synthesis of Compound (19)>

Under an argon stream, 5 ml of dimethylformamide was added to 0.198 g (4.54 mmol) of 55% sodium hydride, and the mixture was stirred at room temperature for 10 minutes. Thereto, 15 ml of a dimethylformamide solution containing 1.190 g (3.78 mmol) of 4-((9-hydroxynonyl)oxy)-2,2,6,6-tetramethylpiperidin-1-oxyl (1-23) synthesized by the above-mentioned reaction was added dropwise over 10 minutes, and the mixture was stirred at room temperature for 3 hours. Furthermore, 15 ml of a dimethylformamide solution containing 2.687 g (3.15 mmol) of 2-(3-bromo-2,2-bis(((1,1,1,3,3,3-hexafluoro-2-(trifluoromethyl)propan-2-yl)oxy)methyl)propoxy)-1,1,1,3,3,3-hexafluoro-2-(trifluoromethyl)propane (1-1) synthesized by the above-mentioned reaction was added in an ice bath, and the mixture was stirred at 60° C. for 20 hours.

Water was added to the reaction solution, and extraction was carried out with ethyl acetate. Thereafter, washing was carried out with a saturated sodium chloride aqueous solution, and the organic layer was dried with magnesium sulfate. The resultant was concentrated under reduced pressure. Subsequently, purification was carried out by silica gel column chromatography (hexane:ethyl acetate=9:1) to obtain the target compound (19) (actual yield: 1.540 g, percent yield: 45%).

When the obtained compound was subjected to mass spectrometry, a peak was confirmed at m/z=1086 (M′). From this result, it was confirmed that the synthesized compound was the compound represented by Formula (19). Furthermore, the degree of purity of the compound represented by Formula (19) confirmed by high-performance liquid chromatography (HPLC) was 98.9%.

### Example 10

### (Synthesis of Compound 20)

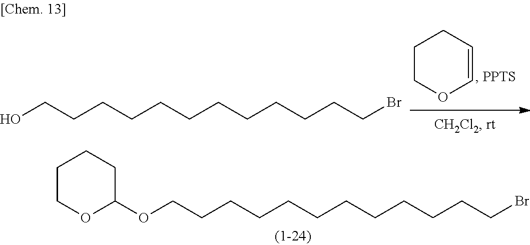

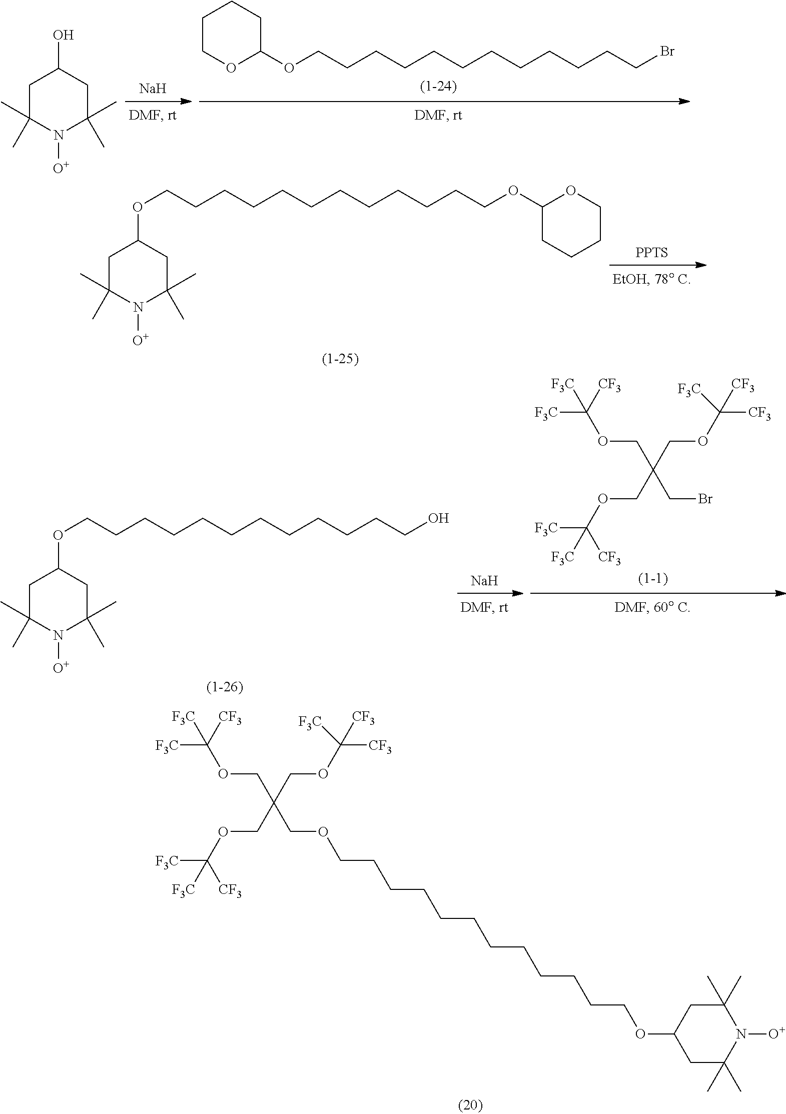

### Synthesis of 2-((12-bromododecyl)oxy)tetrahydro-2H-pyran (1-24)

Under an argon stream, 7.957 g (30.0 mmol) of 12-bromo-1-dodecanol was dissolved in 150 ml of dichloromethane, 3.30 ml (36.0 mmol) of 3,4-dihydro-2H-pyrane was added, and then 1.508 g (6.00 mmol) of pyridinium p-toluenesulfonate (PPTS) was added, and the mixture was stirred at room temperature for 18 hours. The reaction solution was concentrated under reduced pressure. Subsequently, purification was carried out by silica gel column chromatography (hexane:ethyl acetate=9:1) to obtain the target 2-((12-bromododecyl)oxy)tetrahydro-2H-pyran (1-24) (actual yield: 8.280 g, percent yield: 79%).

### Synthesis of 2,2,6,6-tetramethyl-4-((12-((tetrahydro-2H-pyran-2-yl)oxy)dodecyl)oxy)piperidin-1-oxyl (1-25)

Under an argon stream, 10 ml of dimethylformamide (DMF) was added to 0.524 g (12.0 mmol) of 55% sodium hydride, and the mixture was stirred at room temperature for 10 minutes. Thereto, 20 ml of a dimethylformamide solution containing 1.723 g (10.0 mmol) of 4-hydroxy-2,2,6,6-tetramethylpiperidin-1-oxyl was added dropwise over 10 minutes, and the mixture was stirred at room temperature for 3 hours. Furthermore, 20 ml of a dimethylformamide solution containing 4.891 g (14.0 mmol) of 2-((12-bromododecyl)oxy)tetrahydro-2H-pyran (1-24) synthesized by the above-mentioned reaction was added in an ice bath, and the mixture was stirred at room temperature for 18 hours.

Water was added to the reaction solution, and extraction was carried out with diethyl ether. Thereafter, washing was carried out with water, and the organic layer was dried with magnesium sulfate. The resultant was concentrated under reduced pressure. Subsequently, purification was carried out by silica gel column chromatography (hexane:ethyl acetate=9:1) to obtain the target 2,2,6,6-tetramethyl-4-((12-((tetrahydro-2H-pyran-2-yl)oxy)dodecyl)oxy)piperidin-1-oxyl (1-25) (actual yield: 1.631 g, percent yield: 37%).

### Synthesis of 4-((12-hydroxydodecyl)oxy)-2,2,6,6-tetramethylpiperidin-1-oxyl (1-26)

1.631 g (3.70 mmol) of 2,2,6,6-tetramethyl-4-((12-((tetrahydro-2H-pyran-2-yl)oxy)dodecyl)oxy)piperidin-1-oxyl (1-25) synthesized by the above-mentioned reaction, and 0.093 g (0.37 mmol) of pyridinium p-toluenesulfonate (PPTS) were dissolved in 200 ml of ethanol (EtOH), and the mixture was stirred at 78° C. for 3 hours. The reaction solution was concentrated under reduced pressure. Subsequently, purification was carried out by silica gel column chromatography (hexane:ethyl acetate=4:1) to obtain the target 4-((12-hydroxydodecyl)oxy)-2,2,6,6-tetramethylpiperidin-1-oxyl (1-26) (actual yield: 1.108 g, percent yield: 84%).

### <Synthesis of Compound (20)>

Under an argon stream, 5 ml of dimethylformamide was added to 0.163 g (3.73 mmol) of 55% sodium hydride, and the mixture was stirred at room temperature for 10 minutes. Thereto, 15 ml of a dimethylformamide solution containing 1.108 g (3.11 mmol) of 4-((12-hydroxydodecyl)oxy)-2,2,6,6-tetramethylpiperidin-1-oxyl (1-26) synthesized by the above-mentioned reaction was added dropwise over 10 minutes, and the mixture was stirred at room temperature for 3 hours. Furthermore, 15 nil of a dimethylformamide solution containing 2.211 g (2.59 mmol) of 2-(3-bromo-2,2-bis(((1,1,1,3,3,3-hexafluoro-2-(trifluoromethyl)propan-2-yl)oxy)methyl)propoxy)-1,1,1,3,3,3-hexafluoro-2-(trifluoromethyl)propane (1-1) synthesized by the above-mentioned reaction was added in an ice bath, and the mixture was stirred at 60° C. for 20 hours.

Water was added to the reaction solution, and extraction was carried out with ethyl acetate. Thereafter, washing was carried out with a saturated sodium chloride aqueous solution, and the organic layer was dried with magnesium sulfate. The resultant was concentrated under reduced pressure. Subsequently, purification was carried out by silica gel column chromatography (hexane:ethyl acetate=9:1) to obtain the target compound (20) (actual yield: 1.374 g, percent yield: 47%).

When the obtained compound was subjected to mass spectrometry, a peak was confirmed at m/z=1128 (M⁺). From this result, it was confirmed that the synthesized compound was the compound represented by Formula (20). Furthermore, the degree of purity of the compound represented by Formula (20) confirmed by high-performance liquid chromatography (HPLC) was 98.6%.

### Comparative Example 1

1,3-Bis(2,2,2-trifluoro-1,1-bis(trifluoromethyl(ethoxy)-2,2-bis((2,2,2-trifluoro-1,1-bis(trifluoromethyl)ethoxy)methyl)propane (PERFECTA, manufactured by Sigma-Aldrich) represented by Formula (A1) below was prepared.

### Comparative Example 2

Trifluoromethylbenzene represented by Formula (A2) and 4-hydroxy-2,2,6,6-tetramethylpiperidin-1-oxyl free radical (TEMPOL, manufactured by Tokyo Chemical Industry Co., Ltd.) represented by Formula (A3) below were mixed at a molar ratio ((A2):(A3)) of 1:1 to obtain a compound of Comparative Example 2.

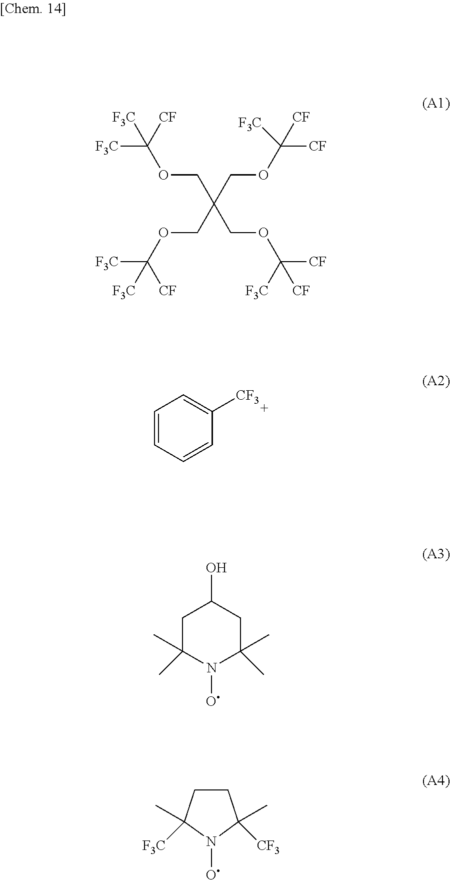

For each of the compounds of Examples 1 to 10, Comparative Example 1, and Comparative Example 2 obtained as above, a ¹⁹F spin-lattice relaxation time (T1) and a ¹⁹F spin-spin relaxation time (T2) were measured by the following method. Table 1 shows the results.

### (Measurement of ¹⁹F Spin-Lattice Relaxation Time (T1))

The compound was dissolved in a deuterated chloroform solution at a concentration of 5 mM, and the longitudinal relaxation time (T1) of the ¹⁹F nucleus was measured using a 500 MHz NMR device by an inversion recovery method under the conditions described below.

### (Measurement Condition)

NMR device: JNM-ECA500 (manufactured by JOEL)
Measurement temperature: 36° C.
Pulse sequence: double_pulse
relaxation_delay: 10 [s]
tau_interval: 4, 3, 2, 1, 0.8, 0.6, 0.4, 0.2, 0.1 [s], 80, 60, 40, 20, 10, 8, 6, 4, 2 [ms]
Accumulated number of times: 128 times

### (Measurement of ¹⁹F Spin-Spin Relaxation Time (T2))

The compound was dissolved in a deuterated chloroform solution at a concentration of 5 mM, and the transverse relaxation time (T2) of the ¹⁹F nucleus was measured using a 500 MHz NMR device by a Carr-Purcell-Meiboom-Gill (CPMG) method under the conditions described below.

### (Measurement Condition)

NMR device: JNM-ECA500 (manufactured by JOEL)
Measurement temperature: 36° C.
relaxation_delay: 10 [s]
tau_step: 1 [ms]
relaxation_delay: 1, 2, 3, 4, 5, 6, 8, 9, 10, 25, 50, 75 [ms], 0.10, 0.25, 0.50, 0.75, 1.00, 1.25, 1.50, 1.75, 2.00 [s]
Accumulated number of times: 128 times

In addition, for each of the compounds of Examples 1 to 10, and the compound of Comparative Example 3 represented by Formula (A4) above, the energy level of a singly occupied molecular orbital (SOMO) was calculated by the method described below. Table 1 shows the results.

### (Calculation of Energy Level of SOMO)

Molecular orbital calculation of the compounds was carried out using Gaussian 09 manufactured by Gaussian, USA. The energy level of the singly occupied molecular orbital (SOMO) was calculated by structure optimization calculation by density functional theory (DFT) using B3LYP as a functional and 6-31+G(d,p) as a basis function.

TABLE 1

| | | T1 | T2 | |
|---|---|---|---|---|
| | | 5 mM/CDCl₃ | 5 mM/CDCl₃ | SOMO |
| Example | Compound | (sec) | (sec) | (eV) |
| 1 | 11 | 0.077 | 0.057 | −0.088 |
| 2 | 12 | 0.070 | 0.049 | −0.085 |
| 3 | 13 | 0.071 | 0.050 | −0.088 |
| 4 | 14 | 0.153 | 0.101 | −0.088 |
| 5 | 15 | 0.145 | 0.092 | −0.085 |
| 6 | 16 | 0.183 | 0.120 | −0.087 |
| 7 | 17 | 0.172 | 0.108 | −0.085 |
| 8 | 18 | 0.428 | 0.274 | −0.087 |
| 9 | 19 | 0.520 | 0.332 | −0.088 |
| 10 | 20 | 0.612 | 0.390 | −0.088 |
| Comparative | A1 | 1.380 | 1.270 | — |
| Example 1 | | | | |
| Comparative | A2, A3 | 1.041 | 0.880 | — |
| Example 2 | | | | |
| Comparative | A4 | — | — | −0.108 |
| Example 3 | | | | |

As shown in Table 1, the compounds of Examples 1 to 10 had shorter ¹⁹F spin-lattice relaxation times (T1) than those of the compounds of Comparative Examples 1 and 2.

In addition, the compounds of Examples 1 to 10 had ¹⁹F spin-spin relaxation times (T2) within an appropriate range. The compounds having a ¹⁹F spin-spin relaxation time (T2) of milliseconds or shorter have fast signal decay and low sensitivity.

Furthermore, the compounds of Examples 1 to 10 had higher energy levels of singly occupied molecular orbitals (SOMO) than that of the compound of Comparative Example 3.

This is because since Comparative Example 3 (compound A4) had only one carbon atom between the 2nd and 5th carbon atoms of the pyrrolidine ring and the fluorine atom, a distance between the nitroxide radical and the fluorine atom was shorter than those of the compounds of Examples 1 to 10. As a result, it is presumed that the nitroxide radical contained in Comparative Example 3 (compound A4) was susceptible to electronic influences from the fluorine atom, and due to the effect of the fluorine atom as an electron-withdrawing group, the energy level of SOMO decreased.

In addition, for each of the compounds of Example 1 (compound 11), Example 3 (compound 13), and Comparative Example 1 (compound A1), a 5 mM chloroform solution was prepared, and T1-weighted images (phantom images) were obtained under the following imaging conditions.

### (Imaging Conditions)

Imaging apparatus: MRI BioSpec 117/11 (manufactured by Bruker Corporation), pulse sequence: RARE VTR
Repetition time: TR=300 ms
Echo time: TE=6 ms
Number of phase encoding=64
Echo train length=1
Flip angle=180°
Accumulated number of times: 16 times
Total imaging time: 5.1 minutes

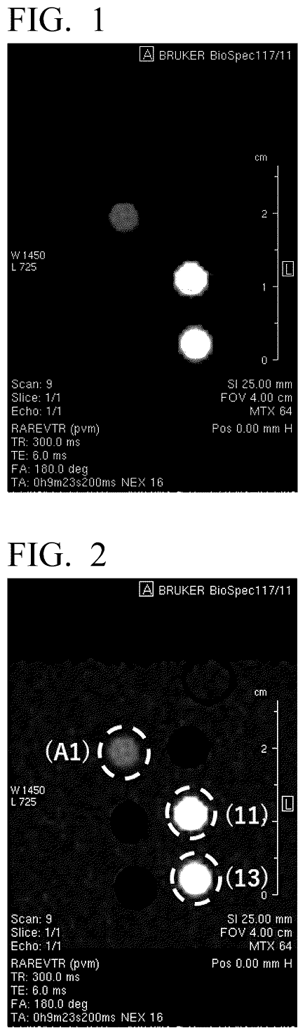

FIG. 1 is a ¹⁹F spin-lattice relaxation time (T1) weighted image of ¹⁹F-MRI in Example 1 (compound 11), Example 3 (compound 13), and Comparative Example 1 (compound A1). FIG. 2 is a ¹⁹F spin-lattice relaxation time (T1) weighted image of ¹⁹F-MRI in Example 1 (compound 11), Example 3 (compound 13), and Comparative Example 1 (compound A1), and is a photograph showing the positions of Examples 1 and 3 and Comparative Example 1 on the image shown in FIG. 1.

In addition, using image processing software (ImageJ), a signal-to-noise ratio (SNR) of each of Examples 1 and 3 and Comparative Example 1 was calculated from the gray values of the positions of Examples 1 and 3 and Comparative Example 1 in the (T1) weighted image shown in FIG. 1. Table 2 shows the results.

TABLE 2

| Compound | SNR |
|---|---|
| 11 | 44 |
| 13 | 27 |
| A1 | 6 |

As shown in FIG. 1 and FIG. 2, the images of Example 1 (compound 11) and Example 3 (compound 13) were brighter than the image of Comparative Example 1 (compound A1).

In addition, as shown in Table 2, it could be confirmed that in Example 1 (compound 11) and Example 3 (compound 13), a large SNR was obtained even with a short imaging time of about 5 minutes, as compared to Comparative Example 1 (compound A1).

From these results, it was shown that by using Example 1 (compound 11) and Example 3 (compound 13) as a contrast agent for MRI diagnosis using fluorine as a detection nucleus, images that are sufficiently applicable to clinical applications can be obtained.

### INDUSTRIAL APPLICABILITY

The fluorine-containing compound of the present embodiment is suitably used as a contrast agent for MRI diagnosis using fluorine as a detection nucleus.

1. A fluorine-containing compound represented by General Formula (1) below, 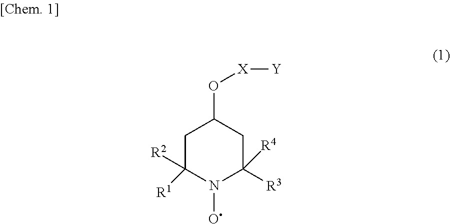
(in General Formula (1), R¹, R², R³, and R⁴ are each independently an alkyl group which has 1 to 10 carbon atoms and which is substituted with a substituent not containing a fluorine atom or is unsubstituted, X is represented by General Formula (2-1) or (2-2) below, and Y is represented by General Formula (3-1) or (3-2) below),
—(CH₂)— (2-1)
—(CH₂)ₘ—O—(CH₂)— (2-2)
(in Formula (2-2), m is an integer of 1 to 12, and —(CH₂)ₘ— in Formula (2-2) is bonded to an oxygen atom in Formula (1)),
—CZ₃ (3-1)
(in Formula (3-1), Z is —CH₂—O—C(CF₃)₃), and
—C(CH₃)Z₂ (3-2)
(in Formula (3-2), Z is —CH₂—O—C(CF₃)₃).

2. The fluorine-containing compound according to claim 1, wherein R¹, R², R³, and R⁴ in General Formula (1) above are each independently an alkyl group which has 1 to 5 carbon atoms and which is substituted with a substituent not containing a fluorine atom or is unsubstituted.

3. The fluorine-containing compound according to claim 1, wherein Y is represented by General Formula (3-1) above.

4. The fluorine-containing compound according to claim 1, wherein X is represented by General Formula (2-2) above.

5. The fluorine-containing compound according to claim 1, wherein R¹, R², R³, and R⁴ in General Formula (1) above are each independently a methyl group or an ethyl group.

6. The fluorine-containing compound according to claim 1, wherein the fluorine-containing compound is used as a contrast agent for magnetic resonance imaging diagnosis using fluorine as a detection nucleus.

7. A contrast agent for magnetic resonance imaging diagnosis which uses fluorine as a detection nucleus, the contrast agent for magnetic resonance imaging diagnosis comprising:
the fluorine-containing compound according to claim 1.

8. The fluorine-containing compound according to claim 2, wherein Y is represented by General Formula (3-1) above.

9. The fluorine-containing compound according to claim 2, wherein X is represented by General Formula (2-2) above.

10. The fluorine-containing compound according to claim 2, wherein R¹, R², R³, and R⁴ in General Formula (1) above are each independently a methyl group or an ethyl group.

11. The fluorine-containing compound according to claim 2, wherein the fluorine-containing compound is used as a contrast agent for magnetic resonance imaging diagnosis using fluorine as a detection nucleus.

12. A contrast agent for magnetic resonance imaging diagnosis which uses fluorine as a detection nucleus, the contrast agent for magnetic resonance imaging diagnosis comprising:
the fluorine-containing compound according to claim 2.
